(19) United States
(12) Patent Application Publication
(10) Pub. No.: US 2009/0230300 A1
(43) Pub. Date: Sep. 17, 2009

(54) RAPID DETECTION OF VOLATILE ORGANIC COMPOUNDS FOR IDENTIFICATION OF BACTERIA IN A SAMPLE

(76) Inventors: Jose Miguel Trevejo, Brighton, MA (US); Shirley Hoenigman, Cambridge, MA (US); James Kirby, Weston, MA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/253,704

(22) Filed: Oct. 17, 2008

Related U.S. Application Data
(60) Provisional application No. 60/999,621, filed on Oct. 19, 2007.
(60) Provisional application No. 61/132,814, filed on Jun. 23, 2008.

(51) Int. Cl. H01J 49/26; C12Q 1/02; G01N 33/497

(52) U.S. Cl. 250/282; 435/29; 73/23.3

(57) ABSTRACT

In various embodiments, the invention relates to a method for identifying the presence of particular bacteria in a sample. The method includes collecting a sample that includes or has been exposed to the particular bacteria and detecting, in the sample, at least one volatile organic compound indicative of the presence of the bacteria.

### CROSS-REFERENCE TO RELATED APPLICATIONS

This application claims the benefit of and priority to U.S. Provisional Patent Application Nos. 60/999,621, filed Oct. 19, 2007, and 61/132,814, filed Jun. 23, 2008, the disclosures of which are hereby incorporated herein by reference in their entirety.

### TECHNICAL FIELD

In various embodiments, the invention relates to methods for detecting one or more volatile organic compounds (herein also referred to as “VOCs” or “organic compounds”) in a sample to determine the presence or absence of one or more bacteria in the sample.

### BACKGROUND

The incidence of bacterial infections, bacterial contaminations, and the spread of drug-resistant bacteria represent growing worldwide health problems. For example, over 8.8 million new cases of tuberculosis are estimated to occur worldwide per year. Recent news has also highlighted the emergence of resistant strains of tuberculosis, which pose a danger to travelers using mass transit, such as commercial airplanes. Rapid detection of the presence or absence of bacteria, of the quantity or bacterial load of bacteria, and of resistant strains of bacteria, such as tuberculosis, is a top priority of health organizations such as the World Health Organization.

Current methods of detecting the presence or absence of bacteria typically include culturing a sample suspected of having bacteria followed by isolation and use of various biochemical tests. These tests may take between 2-21 days to complete depending on how long it takes to isolate and grow the bacteria. Accordingly, while such biochemical testing is relatively inexpensive, it is time consuming to grow and subculture bacteria in a sample to reach the minimal concentration of bacteria needed for testing.

There are PCR-based methods for rapidly detecting bacteria, but these typically are expensive and require advanced laboratory equipment and techniques and various reagents for use. Furthermore, there typically is frequent contamination of samples that precludes use in more resource-limited settings.

By way of example, Mycobacterium tuberculosis (herein also referred to as “TB,” “MTb,” or “M. tuberculosis”) MTb diagnostic standards have not changed significantly in the past century. To the extent that new MTb diagnostics have been developed, they typically are not practical for wide-scale use, for example, in third-world countries. One standard for the diagnosis of active pulmonary tuberculosis is sputum smear microscopy for acid-fast bacilli. If a patient's sputum tests positive for MTb (considered “smear-positive”), they have active pulmonary tuberculosis, are considered highly infectious, and are placed on an exhaustive drug regimen for treatment. However, sputum smear microscopy has low sensitivities and typically requires appropriately trained personnel to accomplish. In fact, it is estimated that sputum smear microscopy at best detects 25-60% of people with active pulmonary tuberculosis. The method also has relatively poor limits of detection as it requires the presence of at least 10,000 MTb bacilli/mL.

Serologic tests do exist for MTb diagnostics, but they continue to undergo development and tend to be more specific for exposure than active disease. Some commercialized tests use immunodominant antigens to detect immunoglobulin classes (like IgG) in an ELISA or dipstick format. Serological tests are estimated to detect one-third to three-quarters of sputum smear-positive cases of MTb. They detect a significantly smaller portion of smear-negative cases with HIV co-infection. In fact, for people infected with both HIV and MTb, serological tests detect less than one third of patients with the active form of the disease.

Phage systems that detect live mycobacteria in liquid cultures using phages that act as indicators by infecting and replicating in MTb cells have been developed. Phage systems appear to be fast, robust and highly sensitive, but little is known about their reproducibility and performance. Phage systems, though highly promising for their speed, robustness, and high sensitivity, typically require, in use, the presence of skilled professionals and may turn out to be very costly. Accordingly, the systems may not lend themselves well to widespread use in developing countries.

Radiometric and fluorescent liquid culture systems, often used in level III laboratories, are highly sensitive, but also may require support of a full microbiology laboratory, typically require relatively long times (1-3 weeks) to generate results, and are relatively expensive to purchase. Radiometric liquid culture systems, though robust and sensitive, require radioactive materials, which therefore typically require special facilities and training for their use. The cost of materials also may be very high and the systems not portable.

Some non-standardized culture systems have also been developed that employ inexpensive reagents and are more suitable for widespread use, but more studies are needed to determine the accuracy of these systems. At the very least, they have demonstrated performance levels comparable to standard diagnostics methods. These non-commercial liquid culture growth detection methods that employ inexpensive reagents may be more suitable for use in developing countries, but they are not yet standardized and thus have not been readily endorsed by TB diagnostics experts. They may benefit from standardization of reagents, packaging, and product support.

Nucleic acid amplification (NAA) systems have been used in industrialized countries for some time, and two systems have been approved by the FDA for MTb detection in sputum. NAA is more sensitive than a smear microscopy test, but less sensitive than culture. NAA, though better than smear microscopy, is expensive and typically requires considerable technical support and quality control. For example, rapid sequencing of bacteria for diagnosis and drug resistance determination (i.e. using PCR) is difficult to make portable and robust for field use. It typically requires significant power and reagents that require special treatment (i.e. cold storage). In addition, even in strictly maintained laboratories there still may be contamination leading to false positive tests. These downfalls make it an unlikely target for a system to be used in high-need (developing) countries.

Another common MTb test is the tuberculin or purified protein derivative (PPD) (PPD skin test), which is the skin test developed for the screening of latent MTb. Additional screens for latent MTb now include new in vitro assays that measure IFN-γ produced by T lymphocytes in whole blood after stimulation from PPDs obtained from MTb, M. avium and M. bovis. Single specific antigens have been used to increase specificity as well. The tuberculin or PPD skin test shares many antigens with a common tuberculosis vaccine, Bacillus Calmette-Guerin (“BCG”), and environmental bacteria so people without latent MTb infection frequently test positive. This approach is further complicated by the need for a clinician to interpret the results and for multiple clinic visits by the patient to obtain the results. The skin test frequently has unreliable results in many patients including those having received a MTb vaccination or those infected with another type of mycobacteria. HIV/AIDS patients frequently test negative when they are also carriers of MTb. Additional skin tests in development (like the one that measures IFN-γ) are more specific, though not perfect, and increasing the specificity of these tests is often at the cost of sensitivity.

Accordingly, rapid point-of-care bacterial detection devices, methods and systems are needed, for example, to screen patients suspected of one or more bacterial infections. Preferably, such devices, methods and systems can be used in the field, for example, for onsite rapid monitoring of the bacterial infections of humans or animals (e.g., in developing countries or any location removed from a laboratory setting), for determining the presence of bacteria in environmental settings, or for testing for bacteria in industrial settings.

### SUMMARY OF THE INVENTION

In various embodiments, the present invention addresses the limitations of current bacterial diagnosis and identification methods by utilizing sensitive detection of certain VOCs to identify the presence of certain bacteria in a sample. This allows, for example, diagnosis of bacterial infection, determination of drug efficacy, and/or diagnosis of drug-resistant bacterial strains in settings outside the laboratory. The bacteria may include, for example, Mycobacterium tuberculosis, Staphylococcus aureus (herein also referred to as “Staph” or “S. aureus”), Klebsiella pneumonia (herein also referred to as “Kleb” or “K. pneumonia”), and/or Escherichia coli (herein also referred to as “E. coli”). Without being bound by theory, certain VOCs are believed to be associated with bacterial metabolism, and therefore may be used to detect viable, recently viable, or growing bacteria isolated in culture or present among a plurality of types of bacteria.

Accordingly, in various embodiments, the present invention is directed to detecting one or more VOCs that are associated with the metabolism, presence, and/or growth of a particular bacteria in order to detect the presence or absence, concentration, state (e.g. viable, growing, etc.) and/or drug resistance status of the bacteria and/or related bacterial strains in a sample. The one or more VOCs may be detected using a portable device, for example a point-of-care device, such as but not limited to a Differential Mobility Spectrometer (“DMS”). The one or more VOCs may be detected directly from a source, for example the exhaled breath of a human or animal (e.g., suspected of having pulmonary tuberculosis (reactivation or primary)), or from gases released from an environmental or industrial source. The VOCs may be generated from a solid or liquid sample, for example from a bodily source, from an environmental source, and/or from an industrial source. The source may be, for example, a tissue or fluid (e.g. urine, sweat, blood, sputum, and/or condensate) from a body, a water or soil sample, and/or an industrial product or waste stream sample.

In one aspect, the invention is directed to a method for identifying the presence or absence of Mycobacterium tuberculosis in a sample. Embodiments of the method include collecting a sample suspected of having Mycobacterium tuberculosis and detecting the presence or absence of one or more volatile organic compound(s) indicative of the presence or absence of Mycobacterium tuberculosis in the sample. The organic compound(s) may be or include methoxybenzene (anisole) (CAS: 100-66-3), 2-butanone (CAS: 513-86-0), methyl 2-ethylhexanoate (for example, a chiral version of methyl 2-ethylhexanoate (CAS: 816-19-3), methyl propionate (CAS: 554-12-1), 2-pentanone, 3-pentanone (CAS: 96-22-0), 2,4-dimethyl-1-heptene, methyl isobutyl ketone, 6-methyl-5-hepten-2-one, dimethylsulfoxide, dimethylsulfide, methyl 2-methylpropionate (CAS: 547-63-7), 1-ethoxy-2-methylpropane (CAS: 627-02-1), 1-ethoxy-butane (CAS: 628-81-9), t-butyl ethyl ether (CAS: 637-92-3), methyl 2-methyl butanoate (868-57-5), isobutanol (CAS: 78-83-1), and/or the aromatic compound represented by the mass spectrum in FIG. 22.

In another aspect, the invention is directed to a method for identifying the presence or absence of Staphylococcus aureus in a sample. Embodiments of the method include collecting a sample suspected of having Staphylococcus aureus and detecting the presence or absence of one or more volatile organic compound(s) indicative of the presence or absence of Staphylococcus aureus in the sample. The organic compound(s) may be or include methanethiol (CAS: 74-93-1), dimethyl sulfide (CAS: 75-18-3), 2,3-butanedione (CAS: 431-03-8), 3-hydroxy-2-butanone (CAS: 513-86-0), butyl acetate (CAS: 123-86-4), and benzeneacetaldehyde (CAS: 122-78-1).

In yet another aspect, the invention is directed to a method for identifying the presence or absence of Klebsiella pneumonia in a sample. Embodiments of the method include collecting a sample suspected of having Klebsiella pneumonia and detecting the presence or absence of one or more volatile organic compound(s) indicative of the presence or absence of Klebsiella pneumonia in the sample. The organic compound(s) may be methanethiol (CAS: 74-93-1), 2-heptanone (CAS: 110-43-0), 2-nonanone (CAS: 821-55-6), and 2-undecanone (CAS: 112-12-9).

In still another aspect, the invention is directed to a method for identifying the presence or absence of Escherichia coli in a sample. Embodiments of the method include collecting a sample suspected of having Escherichia coli and detecting the presence or absence of one or more volatile organic compound indicative of the presence or absence of Escherichia coli in the sample. The organic compound(s) may be or include methanethiol (CAS: 74-93-1), dimethyl disulfide (CAS: 75-18-3), and indole (CAS: 120-72-9).

In any of these aspects, the concentration of one or more of the volatile organic compounds can be detected. The presence or concentration of the detected organic compound(s) in the sample may indicate the presence, concentration, state (e.g. viable, growing, etc.) and/or a phenotypic characteristic (e.g. antibiotic resistance, strain, etc.) of the particular bacteria. In certain embodiments, at least a portion of the one or more organic compounds is unique to a bacteria in the sample (e.g., the bacteria being detected). In certain embodiments, the organic compound(s) are detected in the gas phase. The sample itself may be in the gas phase, for example exhaled breath from an individual, or the gas may be mixed with or generated from a solid or liquid sample, such as a sample grown in culture or medium.

The sample can be obtained from any source, for example from the exhaled breath from an individual. The breath may include body fluid from the individual. Alternatively, the sample can include a fluid, for example body fluid associated with an individual's breath, sputum, blood, urine or pleural fluid. In certain embodiments, the sample includes solid matter, for example tissue or fecal matter. In certain embodiments, the sample is from an environmental or industrial setting, for example soil, water, processed food products and/or process waste streams.

The sample can include bacteria exposed to a candidate therapy for treating the bacteria, for example to detect a therapy-resistant strain of the bacteria. The candidate therapy may be a candidate drug, for example an antibiotic, and the therapy-resistant strain of bacteria may be resistant to the drug. The sample can be analyzed immediately for volatile organic compounds. Alternatively, the sample can be cultured and the headspace of the cultured sample can be analyzed for volatile organic compounds. The detected volatile organic compounds indicative of a bacteria can be the same compounds regardless of culture conditions (e.g., media content), or the compounds can be specific to a bacteria grown in a particular culture condition. In various embodiments, the invention is directed to a method for identifying a bacteria (e.g., Mycobacterium tuberculosis) in a sample. The method includes collecting a sample suspected of comprising the bacteria, culturing the sample using a particular media (e.g. a media that includes propionate), and detecting one or more volatile organic compounds associated with the bacterial metabolism on the particular media that is indicative of a presence of or response to treatment or resistance of the bacteria in the cultured sample.

In various embodiments, the invention is directed to a device for identifying a certain bacteria in a sample. The device can include an input for receiving a sample suspected of certain bacteria and a means for detecting one or more volatile organic compounds indicative of a presence of or response to treatment or resistance of the bacteria in the sample. In one aspect, the device identifies Mycobacterium tuberculosis in the sample and the one or more volatile organic includes methoxybenzene (anisole) (CAS: 100-66-3), 2-butanone (CAS: 513-86-0), methyl 2-ethylhexanoate (for example, a chiral version of methyl 2-ethylhexanoate (CAS: 816-19-3), methyl propionate (CAS: 554-12-1), 2-pentanone, 3-pentanone (CAS: 96-22-0), 2,4-dimethyl-1-heptene, methyl isobutyl ketone, 6-methyl-5-hepten-2-one, dimethylsulfoxide, dimethylsulfide, methyl 2-methylpropionate (CAS: 547-63-7), 1-ethoxy-2-methylpropane (CAS: 627-02-1), 1-ethoxy-butane (CAS: 628-81-9), t-butyl ethyl ether (CAS: 637-92-3), methyl 2-methyl butanoate (868-57-5), isobutanol (CAS: 78-83-1), and/or the aromatic compound represented by the mass spectrum in FIG. 22. In another aspect, the device identifies Staphylococcus aureus in the sample and the one or more organic compounds includes methanethiol (CAS: 74-93-1), dimethyl sulfide (CAS: 75-18-3), 2,3-butanedione (CAS: 431-03-8), 3-hydroxy-2-butanone (CAS: 513-86-0), butyl acetate (CAS: 123-86-4), and benzeneacetaldehyde (CAS: 122-78-1). In another aspect, the device identifies Klebsiella pneumonia in the sample and the one more organic compounds includes methanethiol (CAS: 74-93-1), 2-heptanone (CAS: 110-43-0), 2-nonanone (CAS: 821-55-6), and 2-undecanone (CAS: 112-12-9). In another aspect, the device identifies Escherichia coli in the sample and the one or more organic compounds includes methanethiol (CAS: 74-93-1), dimethyl disulfide (CAS: 75-18-3), and indole (CAS: 120-72-9). In any aspect, the presence of the one or more organic compounds can be indicative of the presence or response to treatment or resistance of the corresponding bacteria in the sample. Alternatively or in addition, the absence of one or more organic compounds can be indicative of the absence or response to treatment or resistance of the corresponding bacteria in the sample.

In certain embodiments, a presence or amount of the bacteria in a sample is identified based on the presence and/or concentration of one organic compound detected in the sample. In certain embodiments, a presence or amount of a bacteria in a sample is determined based on the presence or concentration of two or more organic compounds detected in the sample. In certain embodiments, the presence and/or amount of the bacteria in a sample is identified at various time points, for example following administration of a therapy, so that a change in bacterial burden and/or efficacy of the therapy may be identified.

It is to be understood that the features of the various embodiments described herein are not mutually exclusive and may exist in various combinations and permutations.

### BRIEF DESCRIPTION OF THE DRAWINGS

The foregoing and other objects, aspects, features, and advantages of embodiments of the invention will become more apparent and may be better understood by referring to the following description, taken in conjunction with the accompanying drawings, in which:

FIG. 1A depicts a flow chart for an embodiment of VOC analysis to rapidly diagnose and determine therapy resistance for tuberculosis (and/or other bacteria);

FIG. 1B illustrates an exemplary method for detection of VOCs from the headspace of samples;

FIG. 2A is a schematic block diagram of one embodiment of a DMS;

FIG. 2B is a schematic representation of ions as they pass through the DMS of FIG. 2A;

FIGS. 3A and 3B depict a magnified area of an exemplary DMS output to illustrate the features corresponding to peaks that are present in the MTb sample compared to matched media control, as described in Example 1;

FIG. 4 depicts an exemplary boxplot illustrating separation between a feature that was found to distinguish MTb from a matched media control;

FIG. 5A depicts an exemplary comparison of MTb (line 100) versus propionate media (line 102) total ion chromatograms (“TICs”);

FIG. 5B depicts an exemplary comparison of MTb (line 103) and media control (line 104) TICs (left panel) versus Mycobacterium smegmatis (line 105) and media control (line 106) TICs (right panel);

FIG. 5C shows exemplary results of DMS optimization of identified VOCs. Specifically, a mixture of five identified VOCs was made from purified standards and run on the DMS. The left image shows the previous run parameters and the right shows that the compounds more readily identified in the DMS when the sensor temperature was changed to 40° C.;

FIG. 6A-6E show exemplary overlaid gas chromatography-mass spectrometry (“GC-MS”) chromatograms for an MTb sample and matched media control with FIGS. 6B-6E showing sections of the total chromatograms shown in FIG. 6A;

FIG. 7 depicts an exemplary head-to-tail comparison of National Institute of Standards and Technology (“NIST”) main library mass spectrum of methyl propionate and MTb Peak 1, as part of an analysis to identify methyl propionate as a compound indicative of Mycobacterium tuberculosis in a sample;

FIG. 8 depicts an exemplary chromatogram of replicate injections of methyl propionate in ethanol, as part of an analysis to identify methyl propionate as a compound indicative of Mycobacterium tuberculosis in a sample;

FIG. 9 depicts an exemplary chromatogram of replicate injections of standard 2-butanone in methanol, as part of an analysis to identify 2-butanone as a compound indicative of Mycobacterium tuberculosis in a sample;

FIG. 10 depicts an exemplary head-to-tail comparison of NIST main library mass spectra of 2-butanone and C₁₄H₂₄O, as part of an analysis to identify 2-butanone as a compound indicative of Mycobacterium tuberculosis in a sample;

FIG. 11 depicts an exemplary chromatogram of replicate injections of standard 3-pentanone in ethanol, as part of an analysis to identify 3-pentanone as a compound indicative of Mycobacterium tuberculosis in a sample;

FIG. 12 depicts an exemplary chromatogram of overlaid spectra of MTb and standard 3-pentanone, as part of an analysis to identify 3-pentanone as a compound indicative of Mycobacterium tuberculosis in a sample;

FIG. 13 depicts an exemplary chromatogram of replicate injections of a 2-pentanone standard in methanol, as part of an analysis to identify 2-pentanone as a compound indicative of Mycobacterium tuberculosis in a sample;

FIG. 14 depicts an exemplary head-to-tail comparison of NIST main library mass spectra of 2-pentanone and standard 2-pentanone, as part of an analysis to identify 2-pentanone as a compound indicative of Mycobacterium tuberculosis in a sample;

FIG. 15 depicts an exemplary head-to-tail comparison of NIST main library mass spectra of 2-methyl-N,N-diiso-propylpropanamide and standard 2-pentanone, as part of an analysis to identify 2-pentanone as a compound indicative of Mycobacterium tuberculosis in a sample;

FIG. 16 depicts an exemplary head-to-tail comparison of NIST main library mass spectra of 1-amino-2-methylpyridinium hydroxide and methoxybenzene (anisole), as part of an analysis to identify anisole as a compound indicative of Mycobacterium tuberculosis in a sample;

FIG. 17 depicts an exemplary chromatogram of replicate injections of a methoxybenzene standard in methanol, as part of an analysis to identify anisole as a compound indicative of Mycobacterium tuberculosis in a sample;

FIGS. 18A and 18B show exemplary DMS and chromatographic outputs from DMS and GC-MS, respectively, for three different bacteria, E. coli, S. aureus, and K. pneumonia, and control;

FIGS. 19A and 19B depict exemplary DMS and chromatographic outputs from DMS and GC-MS, respectively, for samples of (i) mixture of E. coli and K. pneumonia, (ii) mixture of S. aureus, E. coli and K. pneumonia, (iii) S. aureus alone, and (iv) media control;

FIG. 20 depicts sections of overlaid exemplary GC-MS chromatograms for E. coli, S. aureus, and K. pneumonia that are relevant to particular VOCs specific for E. coli or K. pneumonia;

FIG. 21 depicts sections of overlaid exemplary GC-MS chromatograms for E. coli, S. aureus, and K. pneumonia that are relevant to particular VOCs specific for S. aureus;

FIG. 22 depicts an exemplary mass spectrometry spectral pattern for a VOC of a volatile aromatic compound detected in MTb cultures regardless of the lipid component in the media;

FIG. 23 depicts a section of overlaid exemplary TICs for Smegmatis, Mtb, and media control showing the peak for the volatile aromatic compound represented in FIG. 22. This compound is detected in MTb cultures cultured with three different lipid components in the media;

FIG. 24A depicts a section of overlaid exemplary TICs of MTb cultures prepared in media with different concentrations of sodium propionate and shows the peak for the volatile organic compound, methyl propionate. FIG. 24B is a bar graph depicting the intensity of methyl propionate signal from TIC peak areas in FIG. 24A; and

FIG. 25 depicts a section of overlaid exemplary TICs of MTb cultures prepared in media containing a mixture of carbon sources.

### DETAILED DESCRIPTION

In various embodiments, the present invention relates to an improved method for identifying bacteria in a sample, and allows for a rapid and accurate diagnosis of certain bacterial infections or contaminations. In addition, embodiments of the invention allow for the determination of efficacy of a drug such as an antibiotic, and/or the diagnosis of certain drug-resistant bacterial strains.

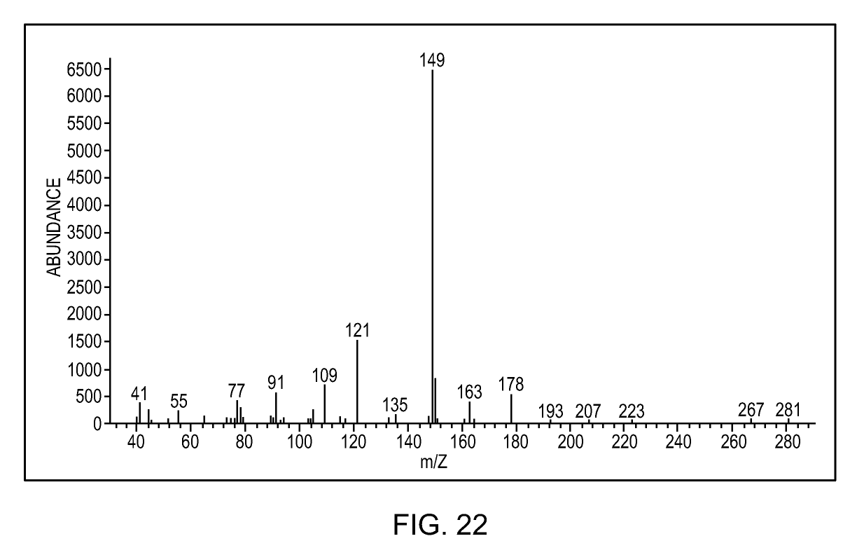

More specifically, MTb may be identified in a sample by detecting the presence or amount of a VOC associated with the presence or amount of MTb, for example a VOC associated with MTb metabolism. These VOCs include, but are not limited to, methoxybenzene (anisole) (CAS: 100-66-3), 2-butanone (CAS: 78-93-3), a chiral version of methyl 2-ethylhexanoate (CAS: 816-19-3), methyl propionate (CAS: 554-12-1), 2-pentanone (CAS: 107-87-9), 3-pentanone (CAS: 96-22-0), 2,4-dimethyl-1-heptene (CAS: 19549-87-2), methyl isobutyl ketone (CAS: 108-10-1), 6-methyl-5-hepten-2-one (CAS: 110-93-0), dimethylsulfoxide (CAS: 67-68-5), dimethylsulfide (CAS: 75-18-3), methyl 2-methylpropionate (CAS: 547-63-7), 1-ethoxy-2-methylpropane (CAS: 627-02-1), 1-ethoxy-butane (CAS: 628-81-9), t-butyl ethyl ether (CAS: 637-92-3), methyl 2-methyl butanoate (868-57-5), isobutanol (CAS: 78-83-1), and/or the aromatic compound represented by the mass spectrum in FIG. 22. Any one of or combination of these VOCs may be used to indicate the presence, concentration, and/or state (e.g. viable, growing, etc.) of the MTb and/or related bacterial strains in the sample. Alternatively, the identification of the absence or concentration below a threshold value of one or more of these VOCs can be used to determine the absence of the bacteria in a sample.

Staph may be identified in a sample by detecting the presence or amount of a VOC associated with the presence or amount of Staph, for example a VOC associated with Staph metabolism. These VOCs include, but are not limited to, methanethiol (CAS: 74-93-1), dimethyl sulfide (CAS: 75-18-3), 2,3-butanedione (CAS: 431-03-8), 3-hydroxy-2-butanone (CAS: 513-86-0), butyl acetate (CAS: 123-86-4), and benzeneacetaldehyde (CAS: 122-78-1). Any one of or combination of these VOCs may be used to indicate the presence, concentration, and/or state (e.g. viable, growing, etc.) of the Staph and/or related bacterial strains in the sample. Alternatively, the identification of the absence or concentration below a threshold value of one or more of these VOCs can be used to determine the absence of the bacteria in a sample.

Kleb may be identified in a sample by detecting the presence or amount of a VOC associated with the presence or amount of Kleb, for example a VOC associated with Kleb metabolism. These VOCs include, but are not limited to, methanethiol (CAS: 74-93-1), 2-heptanone (CAS: 110-43-0), 2-nonanone (821-55-6), and 2-undecanone (112-12-9). Any one of or combination of these VOCs may be used to indicate the presence, concentration, and/or state (e.g. viable, growing, etc.) of the Kleb and/or related bacterial strains in the sample. Alternatively, the identification of the absence or concentration below a threshold value of one or more of these VOCs can be used to determine the absence of the bacteria in a sample.

E. coli may be identified in a sample by detecting the presence or amount of a VOC associated with the presence or amount of E. coli, for example a VOC associated with E. coli metabolism. These VOCs include, but are not limited to, methanethiol (CAS: 74-93-1), dimethyl disulfide (CAS: 75-18-3), and indole (CAS: 120-72-9). Any one of or combination of these VOCs may be used to indicate the presence, concentration, and/or state (e.g. viable, growing, etc.) of the E. coli and/or related bacterial strains in the sample. Alternatively, the identification of the absence or concentration below a threshold value of one or more of these VOCs can be used to determine the absence of the bacteria in a sample.

### 1. Sample Collection and Treatment

Samples can be collected as a solid, liquid, and/or gas and can be treated to determine the presence or absence of one or more VOCs indicative of a particular bacteria being present in the sample. A sample can be analyzed directly for one or more VOCs, for example, from the breath of a patient suspected of having a lung infection. Alternatively, the sample can be cultured in a suitable growth medium to allow growth and metabolism of bacteria in the sample. In certain embodiments, the invention involves taking a sputum sample from an individual and placing it in media, for example, with microfluidics, or in culture, for example, with conventional culturing methods. The bacteria, if present, is stimulated to metabolize. The headspace (gaseous phase) generated as a result of this metabolism may be collected, and may be tested for the presence of at least one metabolite indicative of the bacteria in that growth media.

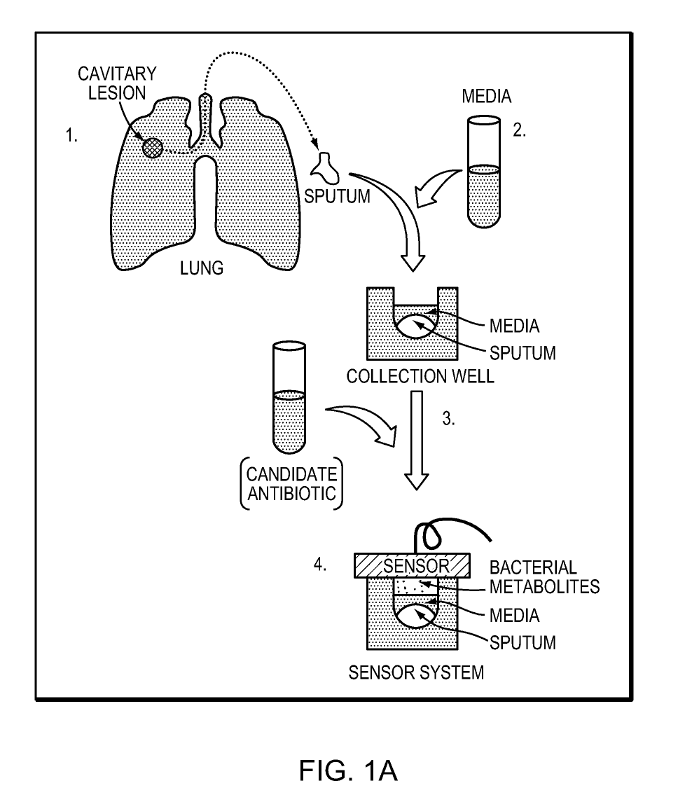

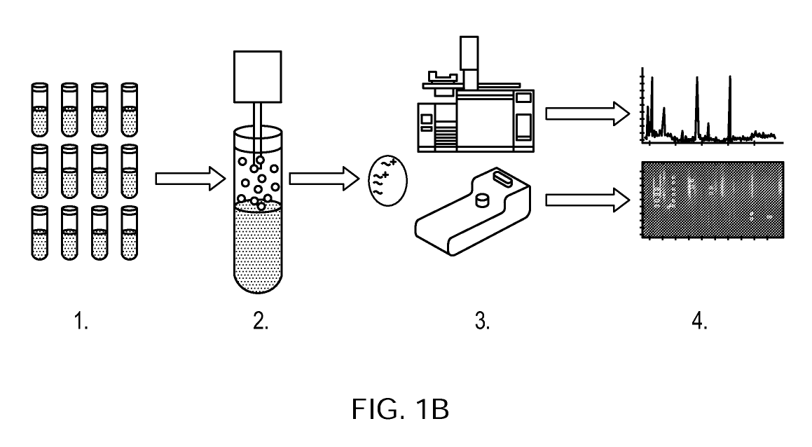

One exemplary embodiment of sample collection and treatment is depicted in FIG. 1A. In the figure: 1. a sputum sample is collected from a subject suspected of harboring unwanted bacteria (e.g. MTb) in the subject's lungs; 2. the sputum sample is transferred to a collection well and provided with medium to stimulate metabolism of bacteria in the sample; 3. optionally, for samples known to include a particular bacteria, such as MTb, a candidate antibiotic can be added to the sputum and media in the collection well to test for efficacy of an antibiotic or bacterial resistance to an antibiotic; and 4. the collection well is sealed with a detection system sensor and a gas headspace is generated (e.g. from bacterial metabolism). One or more volatile organic compounds in the headspace are detected to diagnose bacterial infection or antibiotic efficacy.

A second exemplary embodiment of sample treatment is depicted in FIG. 1B. In the figure: 1. cultured samples are prepared with replicates and matched medium controls; 2. volatile organic compounds in the headspace of the incubated samples are absorbed to concentrating solid phase microextraction (SPME) fiber; 3. the SPME fiber is heated and volatile organic compounds are absorbed into one or more detection systems, for example a GC-MS detection system, a DMS detection system, or a GC-MS/DMS dual system, and data is acquired; and 4. data output is analyzed for features (e.g. one or more VOCs) that distinguish particular bacteria in the sample. When the GC-MS/DMS dual detection is employed, the data from the two systems can be compared to take advantage of database information available from either system.

Samples can be obtained from a variety of sources including biological, environmental and industrial sources. Biological samples can include, for example, exhaled breath directly from an individual or from a breathing machine such as a ventilator, condensate from exhaled breath or a bodily gas, sputum, urine, sweat, blood, plasma, serum, saliva, semen, interstitial fluid, cerebrospinal fluid, dialysate obtained in kidney dialysis, tears, mucus, amniotic fluid, tissue, and fecal matter.

Environmental samples can include, for example, soil, water (e.g., river water, pond water, lake water, groundwater, sewage, drinking water, and swimming pool water), swabs from surfaces in hospitals other public buildings, air samples within buildings or near points of interest (e.g., air ducts), samples from air filters, air conditioning and ventilation systems, or ventilation systems, and dust samples.

Industrial samples can include, for example, manufactured products such as food, drinks, or medicines, waste streams from manufacturing processes, and swabs or gases collected from manufacturing surfaces or spaces.

In certain embodiments, a detector determines the presence or absence, or alternatively the concentration, of the headspace VOCs in the gas phase to determine whether there are viable bacteria in the sample. Accordingly, the sample container is designed to prohibit the release of gas from the sample container or the introduction of ambient gas into the sample container. If an antibiotic known to inhibit or kill the bacteria is also added to the media, then viable bacteria may indicate the presence of resistant bacteria in the sample. Accordingly, potential antibiotics also may be screened using this method.

The bacteria in a sample may be grown in media or in culture, and the media- or culture-grown bacteria may optionally be exposed to a candidate therapy for treating the bacteria, for example a candidate drug such as an antibiotic. Samples can be cultured for any amount of time that allows for generation of VOCs. For example, samples may be cultured for less than 2 hours, 2-4 hours, 4-6 hours, 6-10 hours, more than 10 hours or more than 24 hours. The culture may include any known bacterial culturing media, for example glucose, lipids, short-chain fatty acids, etc., such as propionate, cholesterol, and/or palmitate. In certain embodiments, the VOCs that are detected are specific for a particular bacteria grown on a particular medium. For example, the method can include collecting a sample that includes a particular bacteria, such as MTb, growing the bacteria on a particular medium, such as propionate, and detecting at least one volatile organic compound indicative of the presence of the bacteria in the sample grown on the particular medium. For example, where Mycobacterium tuberculosis is grown on propionate, the organic compound(s) may be or include methyl propionate (CAS: 554-12-1), methyl 2-methylpropionate (CAS: 547-63-7), methyl-2-ethyl hexanoate (816-19-3), and/or the aromatic compound represented by the mass spectrum in FIG. 22. Furthermore, different concentrations of the propionate, and/or mixtures of propionate with other carbon sources, can be included in the growth medium to optimize detection and/or generation of particular VOCs that can be used to detect the presence of or quantitate particular bacteria (e.g., MTb) in a sample. In other embodiments, the VOCs that are detected are the same VOCs regardless of the media components.

### 2. Volatile Organic Compound Detection

The one or more VOCs may be detected using various technologies including, but not limited to: gas chromatography (GC); spectrometry, for example mass spectrometry (including quadrupole, time of flight, tandem mass spectrometry, ion cyclotron resonance, and/or sector (magnetic and/or electrostatic)), ion mobility spectrometry, field asymmetric ion mobility spectrometry, and/or DMS; fuel cell electrodes; light absorption spectroscopy; nanoparticle technology; flexural plate wave (FPW) sensors; biosensors that mimic naturally occurring cellular mechanisms; electrochemical sensors; photoacoustic equipment; laser-based equipment; electronic noses (bio-derived, surface coated); various ionization techniques; and/or trained animal detection.

In various embodiments, the present invention is an improvement over the existing methods for bacterial detection. For example, the sputum smear method of MTb detection is dependent on microscopy to detect the presence of MTb and has a lower limit of detection of 10,000 MTb bacilli/mL and culture methods have a lower limit of detection of 10⁵-10⁶ bacilli/mL. However, embodiments of the present invention do not require microscopy and have lower limits of detection as low as 10³ bacilli/mL. One particular advantage of embodiments of the present invention over standard culturing methods is the amount time for analysis. It typically takes as long as 1-4 weeks to determine TB presence or resistance by current culture methods, whereas both the volatile analysis of sputum and breath samples may yield a result in minutes to hours. To the extent that a sample is cultured to generate VOCs above the culture surface, an adequate culture may be obtained in a matter of hours or days, not weeks as with current methods. In addition, embodiments of the present invention allow for rapid detection of drug resistance if bacterial growth is measured despite the addition of antibiotic. Because embodiments of the present invention utilize the detection of VOCs associated with living bacteria, they also have the ability to increase sensitivity and selectivity. The selectivity derives from the fact that only live bacteria will be actively metabolizing and thus will give a signature, as opposed to serology or other similar techniques that are sensitive but do not distinguish past from present exposure. The increased sensitivity over other methods such as smear microscopy comes from the ability of current ion mass analyzers to detect in the parts per million down to parts per trillion range of sensitivity. Thus, having the known identity of a volatilized compound will enable the exploitation of the increased sensitivities of these mass analyzers.

In certain embodiments, a point-of-care diagnostic tool is used to identify bacterial VOC biomarkers. A point-of-care diagnostic tool, such as a micromachined DMS, preferably is portable and may detect VOCs to low limits of detection. Example 4 below describes a method for identifying the presence of bacteria in a complex clinical sample by detecting one or more VOCs, and/or a spectral pattern of VOCs, using a method that may be portable. Accordingly, in certain embodiments, the present invention includes a library of VOC data and relevant information for a point-of-care diagnostic tool that may be used to identify bacteria in a sample obtained from one or more sources.

### 2A. Differential Mobility Spectrometry

In certain embodiments, the diagnostic tool used to detect the one or more VOCs is a differential mobility spectrometer (Model SVAC, Sionex Corporation, Bedford, Mass.) (“DMS” or “DMS device”). A DMS device can operate at ambient temperature and pressure. A micromachined DMS device has been developed as a portable unit that is mobile and hand-held. The spectrometer produces spectra that differentiates between compounds that may co-elute in a GC-MS system, often yielding an improved ability to identify VOCs in a sample. For matrix-assisted laser desorption ionization/mass spectrometry (MALDI-MS), a statistical model has demonstrated the ability to distinguish between roughly 10 species similar to B. subtilis when the spectral masses are grouped in 1.5 Daltons (Da) ranges. This is due to roughly the same number of proteins per unit-mass interval. Recent data also suggests a 75% correct identification rate using MALDI-MS with no false positives. However, with the DMS technology, even larger numbers of species may be easily distinguished, as the spectra may be more easily deconvoluted than those of MS due to differing ion mobilities.

DMS devices are quantitative and can have extremely sensitive detection limits, down to the parts-per-trillion range. DMS technology uses the non-linear mobility dependence of ions on high strength RF electric fields for ion filtering, and operates in air at atmospheric pressure. DMS technology enables the rapid detection and identification of compounds that typically cannot be resolved by other analytical techniques. DMS devices scale down well, allowing miniaturization of the analytical cell using MicroElectroMechanical (MEMS) fabrication, while preserving sensitivity and resolution. These and other advantages of a DMS device make it attractive as a quantitative detector that is sufficiently low in cost to be practical for use in the field, for example in point-of-care diagnostics in clinical settings.

Conceptually, the operating principle of a DMS device is similar to that of a quadrupole mass spectrometer, with the significant distinction that it operates at atmospheric pressure so it measures ion mobility rather than ion mass. Mobility is a measure of how easily an ion travels through the air in response to an applied force, and is dependent on the size, charge and mass of the ion. A DMS spectrometer acts as a tunable ion filter.

To perform a measurement, a gas sample is introduced into the spectrometer, where it is ionized, and the ions are transported through an ion filter towards the detecting electrodes (Faraday plates) by a carrier gas. The DMS device can separate chemical components of a substance based on differing ion mobilities.

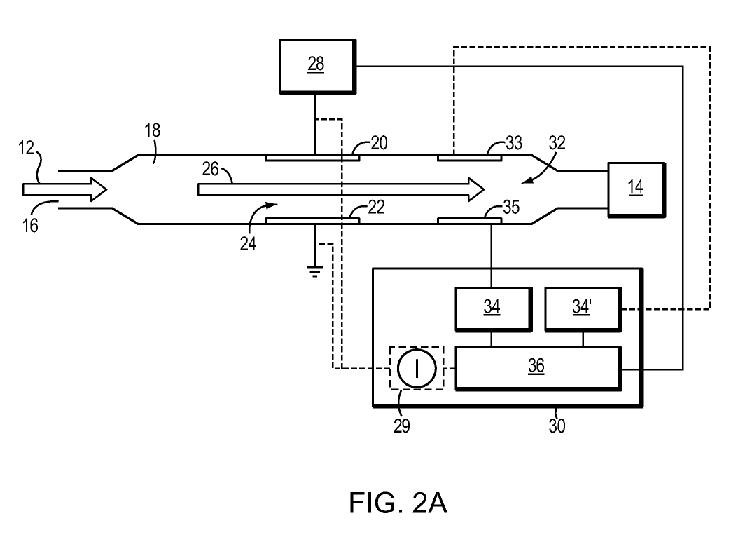

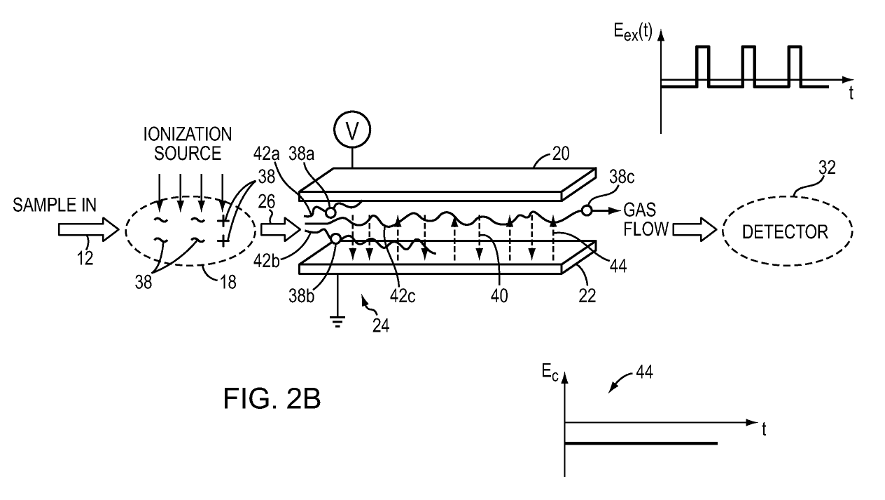

As shown in FIGS. 2A and 2B, certain embodiments of the DMS device operate by introducing a gas, indicated by arrow 12, into ionization region 18. The ionized gas follows flow path 26 and passes between parallel electrode plates 20 and 22 that make up the ion filter 24. As the gas ions pass between plates 20 and 22, they are exposed to an electric field between electrode plates 20 and 22 induced by a voltage applied to the plates. In certain embodiments, the electric field produced is asymmetric and oscillates in time.

As ions pass through filter 24, some are neutralized by plates 20 and 22 while others pass through and are sensed by ion detector 32. In certain embodiments, the detector 32 includes a top electrode 33 at a predetermined voltage and a bottom electrode 35, typically at ground. The top electrode 33 deflects ions downward to the bottom electrode 35. However, either electrode may detect ions depending on the ion and the voltage applied to the electrodes. Moreover, multiple ions may be detected by using top electrode 33 as one detector and bottom electrode 35 as a second detector.

The electronic controller 30 may include, for example, an amplifier 34 and a microprocessor 36. Amplifier 34 amplifies the output of detector 32, which is a function of the charge collected by electrode 35 and provides the output to microprocessor 36 for analysis. Similarly, amplifier 34′, shown in phantom, may be provided where electrode 33 is also utilized as a detector.

Referring now to FIG. 2B, as ions 38 pass through alternating asymmetric electric field 40, which is transverse to gas flow 12, electric field 40 causes the ions to “wiggle” along paths 42a, 42b and 42c. Time varying voltage V is typically in the range of +/−(1000-2000) volts and creates electric field 40 with a maximum field strength of 40,000 V/cm. The path taken by a particular ion is a function of its mass, size, cross-section and charge. Once an ion reaches electrode 20 or 22, it is neutralized. A second, bias or compensation field 44, typically in the range of +/−2000 V/cm due to a +/−100 volt dc voltage, is concurrently induced between electrodes 20 and 22 by a bias voltage applied to plates 20 and 22 also by voltage generator 28, FIG. 2A, in response to microprocessor 36 to enable a preselected ion species to pass through filter 24 to detector 32. Compensation field 44 is a constant bias that offsets alternating asymmetric field 40 to allow the preselected ions, such as ion 38c to pass to detector 32. Thus, with the proper bias voltage, a particular species of ion will follow path 42c while undesirable ions will follow paths 42a and 42b to be neutralized as they encounter electrode plates 20 and 22.

The output of DMS spectrometer 10 is a measure of the amount of charge on detector 32 for a given bias electric field 44. The longer the filter 24 is set at a given compensation bias voltage, the more charge will accumulate on detector 32. However, by sweeping compensation voltage 44 over a predetermined voltage range, a complete spectrum for sample gas 12 can be achieved. The DMS device according to certain embodiments of the invention typically requires less than thirty seconds and as little as one second to produce a complete spectrum for a given gas sample. By varying compensation bias voltage 44, the VOC to be detected can be varied to provide a complete spectrum of the gas sample.

In certain embodiments, the DMS device includes an ion flow generator for propelling the ions 38 generated by the ionization source through the asymmetric electric field 40 created by the ion filter 24 and toward the detector 32. Opposed electrode pairs may create the ion flow generator, for example ring electrode pairs and/or planar electrode pairs. Also, the ion flow generator may create a longitudinal electric field in the direction of the intended ion travel, toward, for example, the detector 32. The strength of the longitudinal electric field can be constant in time or space and can vary with time and space. The longitudinal electric field can propel ions 38 through asymmetric electric field 40. In certain embodiments, the DMS device includes a gas chromatography column. In others embodiments, the DMS device is coupled to a gas chromatography column.

In certain embodiments, the ion filter 24 is disposed in an analytical gap, downstream from the ionization source, for creating an asymmetric electric field to filter ions generated by the ionization source. DMS devices are described in greater detail in U.S. Pat. No. 6,512,224 entitled “Longitudinal Field Driven Field Asymmetric Ion Mobility Filter and Detection System,” and U.S. Pat. No. 6,495,823 entitled “Micromachined Field Asymmetric Ion Mobility Filter and Detection System,” U.S. Pat. No. 6,815,669 entitled “Longitudinal Field Driven Ion Mobility Filter and Detection System,” which are hereby incorporated herein by reference in their entirety.

### 2.B. Data Libraries

In certain embodiments, the diagnostic device (e.g. a GC-MS device, a DMS device, a GC-MS/DMS dual system, or any of the devices described above) can include electronics capable of storing a library of information about VOCs that are indicative of various microorganisms. Alternatively, the electronics can allow for connectivity to one or more remote databases. In the library or databases, previously collected and/or known VOC data, e.g. GC-MS and/or DMS spectral patterns, may be associated with certain microorganisms and/or include associations with other relevant information. Other relevant information may include, for example, information about culturing conditions (e.g. media, media components, temperature, and/or headspace gases above the culture) of a sample that undergoes a culturing step, information about the bodily source of a sample obtained from the body (e.g. tissue type or bodily fluid type), information about the environmental source of a sample obtained from the environment (e.g. soil type or liquid type), and information about an industrial setting that is the source of the sample (e.g. likely contaminants and nutrient sources). Such information may be used in a portable device for the rapid delivery of results that identify particular microorganisms in a sample.

For example, in Example 4 below, DMS data was compared with data collected from simultaneous detection of VOCs using GC-MS. The comparison of the data from the DMS detector with the GC-MS data allows for the generation of a data library for a portable device that uses DMS detection.

### EXAMPLES

Example 1 describes a method for identifying a single bacteria type, MTb, from its matched medium and from other mycobacterial strains, as well as a method for identifying VOCs indicative of MTb in a sample. Example 2 also describes exemplary methods for identifying and detecting exemplary volatile organic compounds in a sample. Specifically, Example 2 describes a method for the identification, using mass spectrometry analysis, of chromatographic peaks of volatile organic compounds indicative of MTb in a sample. Example 3 describes an exemplary method for identifying single bacteria types, Staph, Kleb, and E. coli, from their matched medium, as well as an exemplary method for using a point-of-care diagnostic tool to identify particular bacteria in a sample. Example 3 also describes generating a library of data for the tool to facilitate rapid identification of bacteria in a sample. Example 4 describes experiments showing that certain VOCs consistently may be associated with a particular bacteria across different culture media or, alternatively, may be associated with a particular bacteria cultured with a particular type, concentration or mixture of media component(s). Example 5 shows that one or more VOCs may be used to identify the state (e.g. viable, growing, etc.) of a particular bacteria in the sample, for example bacteria exposed to an antibiotic.

### Example 1

To evaluate the ability of the method to detect bacterial VOCs, two studies with MTb as well as two additional mycobacteria, M. smegmatis (MSmeg) and M. avium (MAC) as control strains, were conducted. In the first study, two strains of MTb and one strain of MAC were cultured at low concentrations (˜10̂5 bacilli/mL) in Bactec™ 12B media. This investigation confirmed that bacterial identification was possible at these low bacilli concentrations and confirmed that the method is capable of identifying: a) each bacterium from its matched medium; b) one MTb strain from another MTb strain; and c) MTb from a mycobacterium control strain (MAC). In the second study, the quantity of VOCs extracted from the headspace was increased to confirm that the compounds could be identified by mass spectrometry.

1a. Culture and Data Acquisition Methods

FIG. 1B as described above illustrates the general method that was used to acquire the data for the two studies. Bacteria were grown in Bactec™ bottles in eight replicates per treatment. Both strains were harvested at selected growth indices, as determined from the Bactec™ machine and their headspace was extracted using SPME fiber, which adsorbs and concentrates only the VOCs. The VOCs were desorbed from the SPME fibers into a gas chromatograph and then re-concentrated on a cryogenic trap. VOCs were separated chromatographically and the eluent stream was split post-column. Roughly half of the eluent was diverted into a quadrupole mass spectrometer (MS) while the other half was transferred to a DMS device. MS was used for compound identification and to ensure peak correlation between the two detectors.

In the second study, the concentration of the bacilli was 10⁸/mL or roughly 10³ times greater than that of the first study, and the headspace incubation time was increased to 24 hours. Moreover, one strain of MTb and one control strain (MSmeg) were cultured in Middlebrook 7H9 media using a short-chain fatty acid (propionate) as the carbon source instead of dextrose and glycerol. Fatty acid metabolism was expected to produce more physiologically relevant VOCs, as several studies suggest that MTb likely utilizes lipids in vivo. A second culture of MTb was prepared in Middlebrook 7H9 media with dextrose and glycerol. The headspace VOCs were extracted during the exponential growth phase for each culture using the same procedure previously described.

The data generated from the DMS detection was analyzed using two-class comparison between each bacterium and its matched media control as well as between MTb and the control mycobacterial strains. Feature selection was accomplished based on peaks that were different between averaged data of the eight replicates. Features were then individually investigated to confirm that they were present or absent between the two classes. Once these features were identified they were used to determine classification percentage.

1b. Results—DMS Distinguishes MTb from Media and Control Mycobacterium

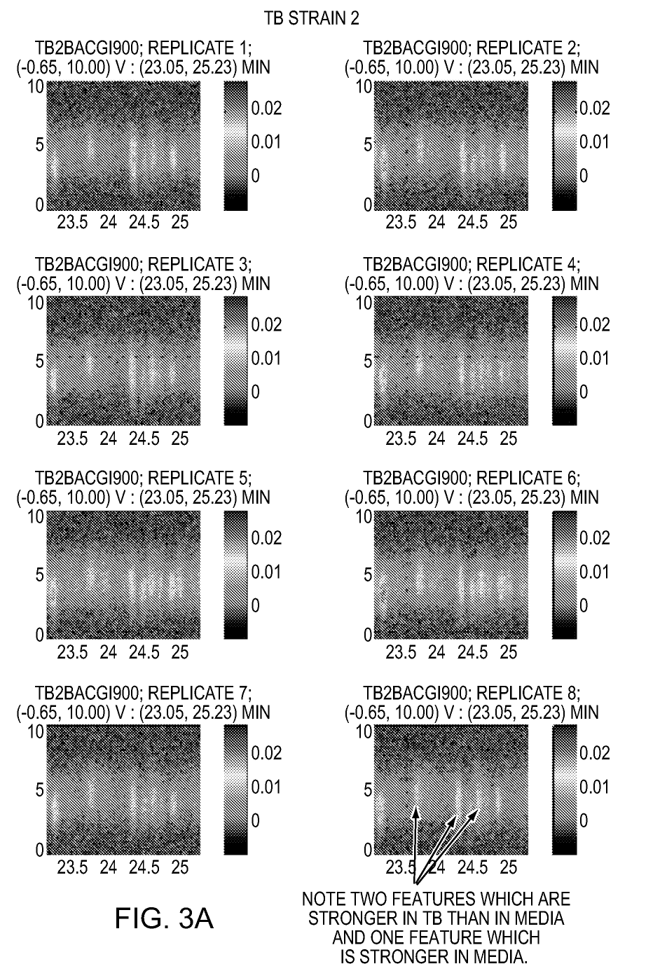

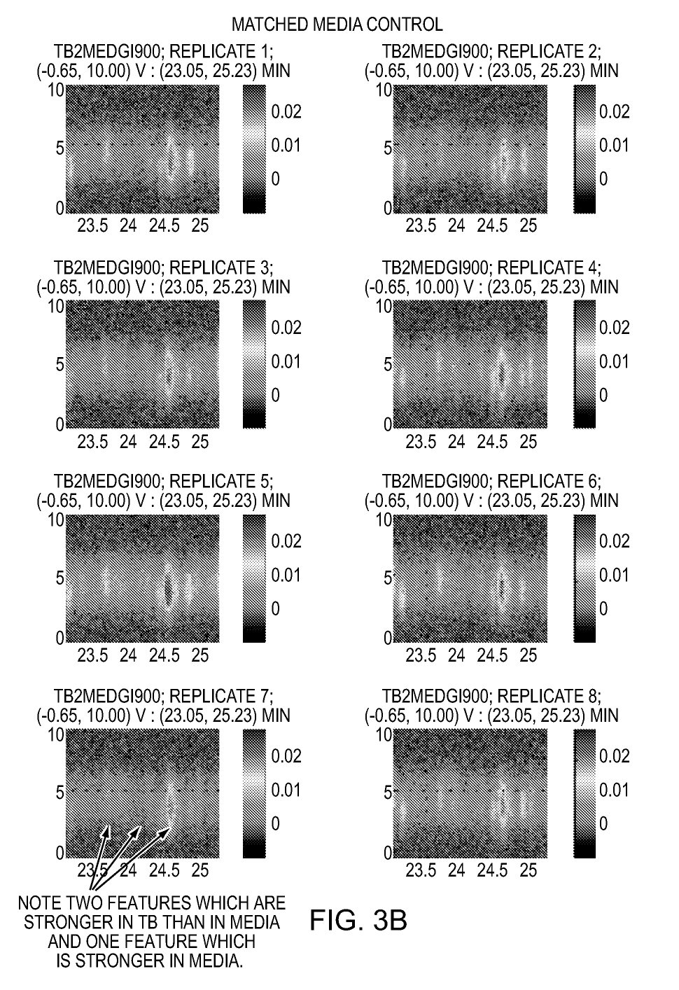

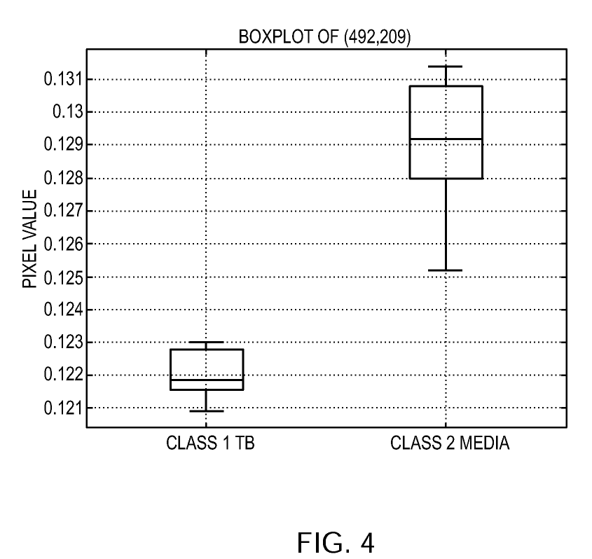

Positive features corresponding to new or more intense peaks present in the MTb versus the matched media control are ideal features, as they represent metabolite production from the MTb and thus may be present in the lungs of patients during active infection. A representative set of features is shown in FIGS. 3A and 3B, which depict a magnified area of the DMS output to illustrate the features corresponding to peaks that are present in the MTb sample compared to matched media control. Selected features were then used for class separation between MTb and media or between MTb and control strains. A representative boxplot of a selected feature that illustrates differentiation between MTb Strain 1 and its matched media is shown in FIG. 4. This was one of the features found for MTb strain 1 at a growth index of 900. Two or three features were used to discriminate between the classes of bacteria using standard algorithms of either K-nearest neighbor or support vector machines. A summary of the resulting classification efficiencies is shown in Table A.

TABLE A

| | Growth Index | | |
|---|---|---|---|
| Comparison | (~bacilli/mL) | % Correct Classification | |
| TB Strain 1 vs. Media | 10 (10⁵) | 81.25 | 87.5 |
| TB Strain 1 vs. Media | 500 (10⁶) | 93.3 | 86.7 |
| TB Strain 1 vs. Media | 900 (10⁶-10⁷) | 100 | 100 |
| TB Strain 1 vs. MAC | 500 | 100 | 100 |
| TB Strain 2 vs. Media | 10 | 100 | 100 |
| TB Strain 2 vs. Media | 500 | 100 | 100 |
| TB Strain 2 vs. Media | 900 | 100 | 100 |
| TB Strain 2 vs. MAC | 500 | 100 | 100 |
| TB Lipid vs. Media | OD⁵⁶⁰ = 0.5 (5 × 10⁸) | 100 | 87.5 |

Specifically, Table A shows the results of two-class comparisons of Mycobacterium tuberculosis and matched media or Mycobacterium avium complex using DMS analysis of volatile organic compound-containing headspace. Bacteria were cultured at an initial concentration of 10⁵ bacilli/mL. The last row was a separate experiment performed in lipid media; the extraction and analysis procedures for these data were the same. After headspace extraction using SPME fibers and analysis by DMS, the MTb, MAC and matched media control data were analyzed by transforming the data with a two-dimensional discrete wavelet transform (2D-DWT). Wavelets may be considered as orthogonal features that are localized in compensation voltage and time; they also have utility in smoothing, denoising, and baseline removal tasks. Baseline removal was then executed on the signal by setting the wavelet approximation coefficients to zero. Smoothing was effectively performed on the signal by removing from consideration features derived from the highest level of detail coefficients. The remaining coefficients were ranked using the Fisher Linear Discriminant. Redundant coefficients were eliminated automatically. Ultimately, features were validated for use in classification and for consideration as biomarkers by inspection. Selected features were then submitted to a K-Nearest Neighbor classifier and a Support Vector Machine Classifier using cross-validation.

Table A confirms that features were detected in MTb that gave high separability between MTb and the media background as well as between MTb and MAC. A subset of these features was identical between MTb strain 1 and strain 2, suggesting that they may correspond to VOCs from MTb. It should be noted that the MS detected little to no corresponding signal for these features, suggesting that DMS has lower detection limits and/or different selectivity than MS in this experiment. Nevertheless, this study confirms that DMS may be used to identify MTb in a sample by detecting VOCs indicative of MTb, for example MTb VOCs, with high separation and low bacterial detection limits, for example at a bacilli concentration as low as 10⁵ bacilli/mL.

1c. Results—Mass Spectrometry Identification of MTb Volatile Organic Compounds

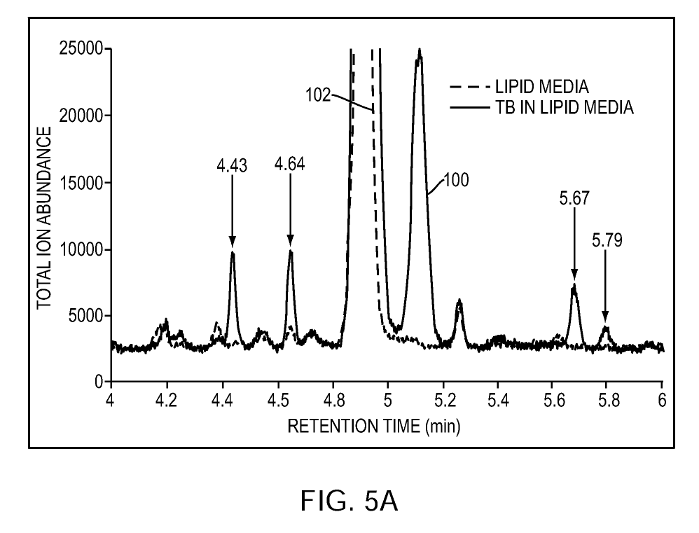

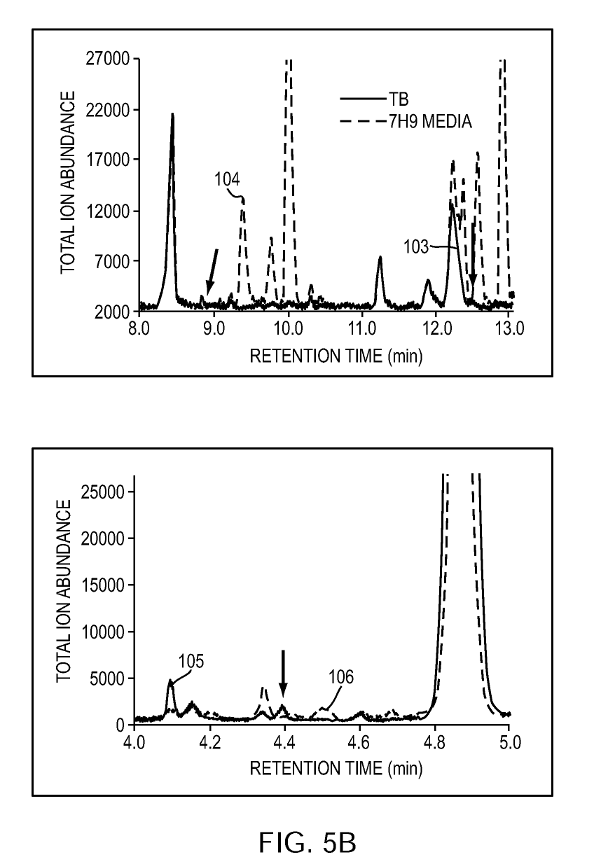

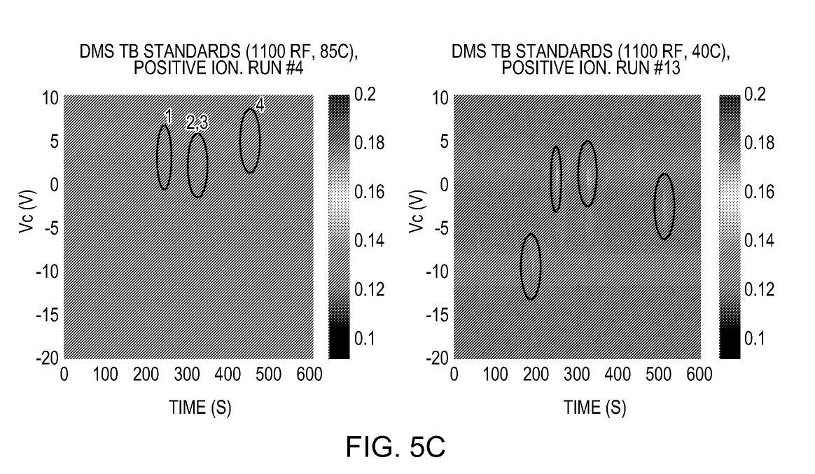

In the second study, the quantity of VOCs extracted from the headspace was increased. Representative TICs of MTb and propionate media are shown in FIG. 5A. FIG. 5A depicts a comparison of MTb (line 100) versus propionate media (line 102) total ion chromatograms. The headspace of standard solutions prepared from pure compounds was extracted using SPME and the resulting chromatographic and mass spectral data confirmed VOCs indicative of MTb. Seven peaks unique to MTb were identified from the MTb cultured with media that included propionate. Four of the seven peaks are shown in FIG. 5A (arrows and retention time). Exemplary methods for identifying and further characterizing selected VOCs are described more fully in Example 2, below.

When the same MTb strain was grown in 7H9 media using dextrose/glycerol as the carbon source, only two unique MTb peaks were identified in the chromatogram (see arrows in FIG. 5B, left panel). FIG. 5B depicts a comparison of MTb (line 103) and control media (line 104) TICs (left panel) versus Mycobacterium smegmatis (line 105) and control (line 106) TICs (right panel).

For M. smegmatis cultured in lipid phase, only one unique peak was observed for the bacteria relative to the matched media. This peak was also observed among the MTb VOCs in lipid media, which indicates that the two bacteria share at least one common metabolite and perhaps a metabolic pathway. Collectively, these results confirm a unique metabolic profile that allows for discrimination between different mycobacteria and different growth environments. The identification of VOCs unique to MTb also affords the opportunity to optimize various detection methods, for example DMS sensor detection of such compounds in breath. Specifically, the RF and V_{C} electrical fields may be maintained at fixed voltages that preferentially select compounds of interest as they elute from the chromatographic column so that the maximum number of desired ions is transmitted to the detector to produce a concomitant increase in signal. Using this approach, the methods described herein may facilitate detection of these compounds present in the breath of individuals exposed to various bacteria.

Another example of such optimization is shown in FIG. 5C for a mixture containing five known VOCs that were identified by GC-MS. A mixture of five identified VOCs was made from purified standards and run on the DMS. The left side of FIG. 5C shows the compounds run on the DMS using previous run parameters and the right shows the compounds using an optimized parameter of temperature. As shown in FIG. 5C, the compounds are more readily identified in the DMS when the sensor temperature was changed to 40° C. These data were collected from sensor temperature/dispersion voltage combinations of 85° C./1100V (FIG. 5C left) and 40° C./1100V (FIG. 5C right). When the dispersion voltage was held constant, Peak 5 around 200 seconds is not observed at the higher temperature (85° C.). However, as the sensor temperature was lowered to 40° C., the fifth metabolite peak was observed with an increase in peak intensity for all analytes in the mixture. In these two sets of experiments the dispersion was held constant. Other temperature/dispersion voltage combinations may increase peak separability for Peaks 2 and 3.

1d. Results and Discussion

In summary, these results confirm that particular bacteria can be distinguished from media or other bacteria with high classification efficiency. Furthermore, tandem detection systems enable identification of at least 15 compounds indicative of MTb. This identification allows for optimization of detection, for example DMS detection, to increase the sensitivity of detection for VOC biomarkers, for example, in a breath or sputum sample.

### Example 2

Data from three sets of samples were collected and analyzed. The three sets of samples included M. tuberculosis in Middlebrook 7H9 with added propionate (referred to as lipid phase), M. tuberculosis in Middlebrook 7H9, and M. smegmatis in the lipid phase. Media alone and air extraction served as controls for each data set. The volatiles from the headspace for each bacteria sample was extracted using SPME fibers during the exponential growth phase by exposure of the fiber to the headgas for 30 minutes, and the OD values were 0.48, 0.48, and 0.63, respectively, for MTb in lipid, MTb in 7H9, and M. smegmatis. The volatiles from the headspace for the non-bacteria samples was extracted in the same manner as the bacteria above and it should be noted the media was incubated for the same period of time in the same vessels and conditions. The relevant samples are summarized in Table B.

TABLE B
Mass Spectrometry analysis of samples analyzed
for the MTb Lipid Study

| Conditions | Sample Identification |
|---|---|
| M. tuberculosis + 7H9 + Propionate | 13_4, 13_6, 13_9, 13_10, |
| | 14_4, 14_6, 14_7, 15 _5, 15_7 |
| Media | 13_3, 13_5, 13_11, 13_12, |
| | 14_3, 14_5, 15_4, 15_6 |
| Air extraction | 13_7, 15_3 |
| M. tuberculosis + 7H9 | 22_3, 22_6, 22_7, 22_8, 23_4, |
| | 23_7, 25_5, 25_7 |
| Media | 22_4, 22_5, 23_3, 23_6, 25_3, |
| | 25_4, 25_6, 25_8 |
| Air extraction | 23_5 |
| M. smegmatis + 7H9 + Propionate | 26_3, 26_5, 27_3, 27_5, |
| | 28_9, 28_10, 28_11 |
| Media | 27_4, 27_6, 28_3, 28_4, 28_5, |
| | 28_6, 28_7, 28_8 |
| Air extraction | |

For each condition, multiple gas extraction samples for each condition were collected and identified with a unique identifier, e.g. 13_{—}4. For example, for the MTb in lipid phase, nine replicates were listed. For M. smegmatis, no air extraction fiber was identified.

2a. Mass Spectrometry Identification of Peaks Associated with M. tuberculosis in Lipid Phase

The replicates of air, lipid phase, and MTb samples were examined, and a comprehensive list of peak retention times and identifications for air, the propionate media, and MTb in lipid media were collected. Select peaks were compared to known compounds using the ChemStation NIST library. Generally, the highest percent match was chosen for a peak, provided the percent match was ≧30%. While this may not be an acceptable threshold value in MS peak identification, where a match of ≧70% is commonly used, most chromatographic peaks in these data did not have substantial peak intensity (i.e., >10000 for total ion abundance). However, without any knowledge of what analytes were present in the MTb headspace, it was reasonable to employ a lower threshold percent match.

The mean, standard deviation (sd), and percent relative standard deviation (% rsd) were calculated for each peak's retention time. For a robust chromatographic system, ≦2.0% rsd of sample analytes is desirable, with ≦1.0% preferable. Evaluation of the results confirmed reproducibility in retention times for both the lipid media and the MTb chromatographic retention times. A maximum % rsd of 1.7% was calculated for the first peak (average retention time=3.18 min) of the MTb samples while the corresponding lipid media peak (average retention time=3.19 min) had a relative error of 0.71%. In all cases, this was a broad siloxane peak, and based on its short retention time, broad peak width, and lack of analytical usefulness, it was disregarded. All other remaining peaks had relative error values between 0.064-0.40% rsd for MTb and between 0.035-0.36% rsd for the lipid media. Finally the total number of peaks in the lipid media and the MTb chromatograms was determined using the criterion that a peak must appear in at least three of the eight chromatograms which represent each of the eight replicates of a sample treatment. Thus, the lipid media was found to have 39 peaks while the MTb had 38 peaks.

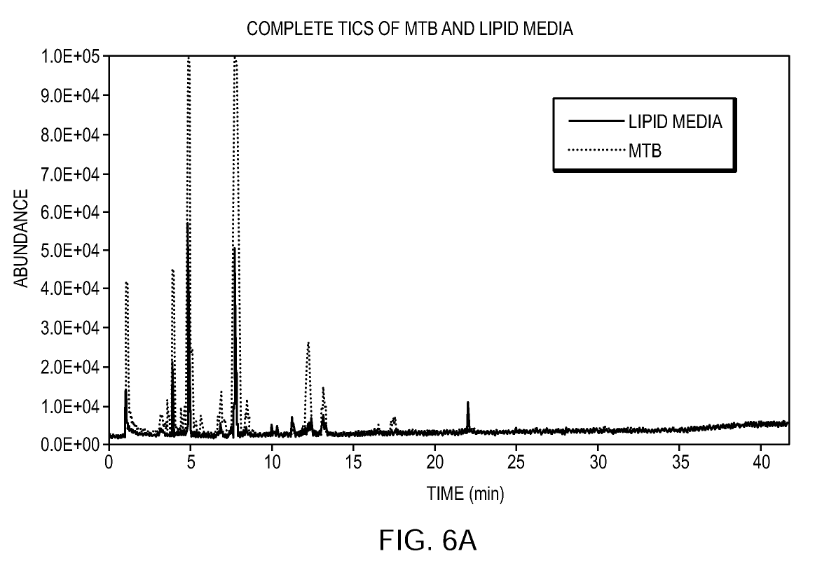

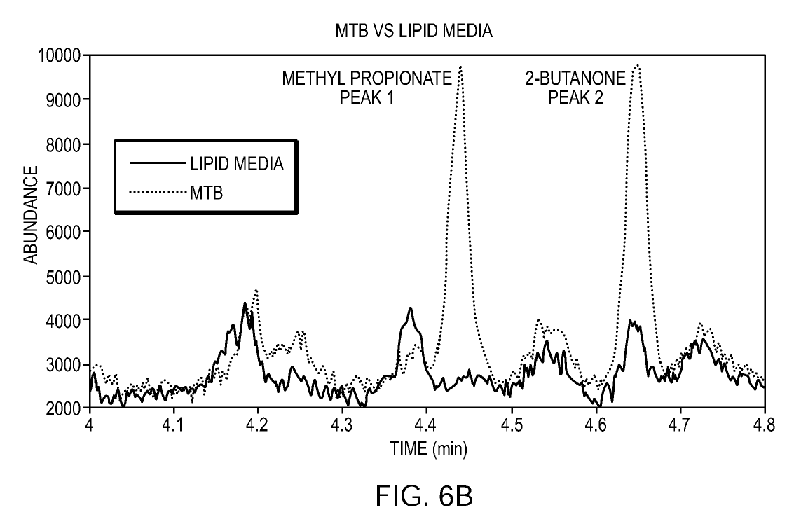

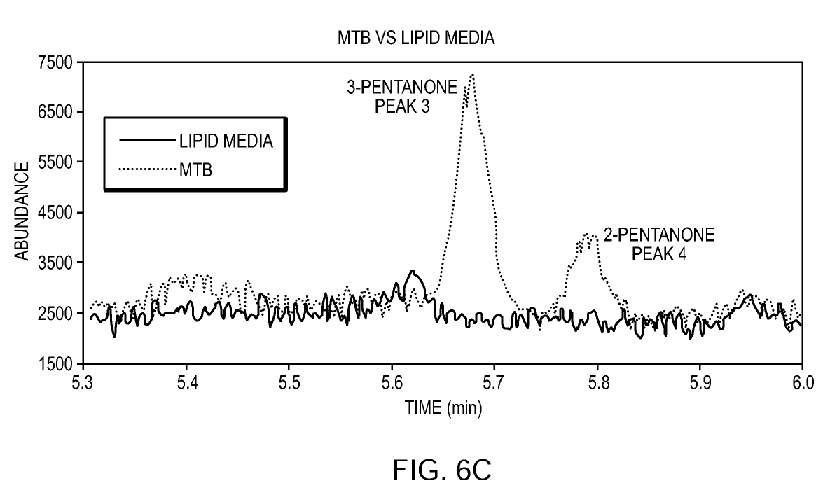

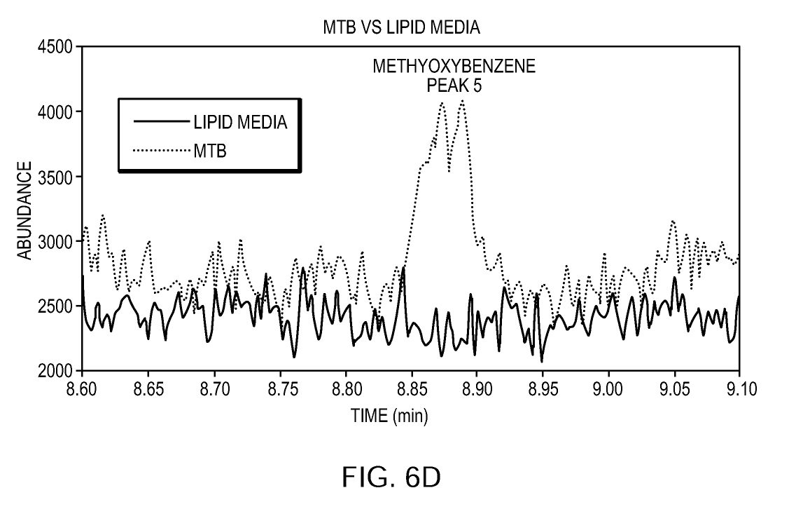

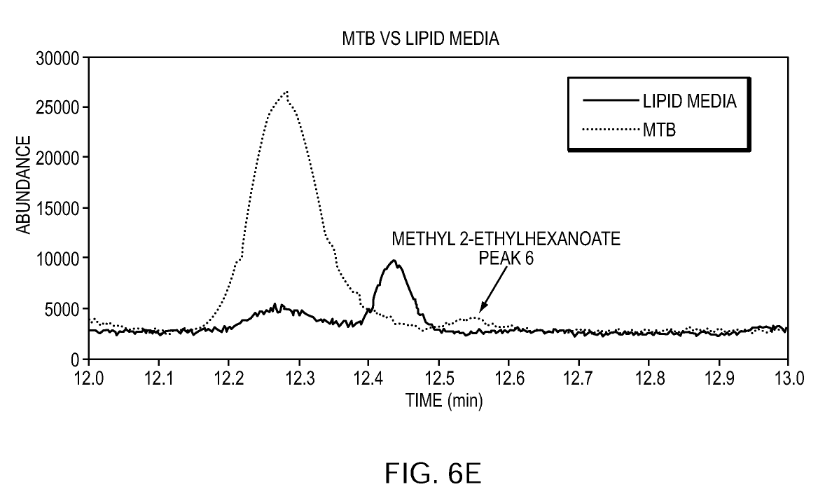

With peak identification and reproducibility assessed, TICs were overlaid for MTb in lipid phase versus the lipid phase alone. To simplify comparison, only bacteria and media that were analyzed via GC-MS on the same day were compared. This minimized day-to-day variability in analyte retention inherent in any chromatographic technique. Based on the confirmed reproducibility of the analyte retention times, no interpretation error was introduced by this choice. FIGS. 6A-6E show exemplary MTb chromatograms overlaid with chromatograms of matched media controls. Six exemplary peaks indicative of MTb are each identified in FIGS. 6B-6E by the peak number and the identity of the compound as determined below.

TABLE C
Average Retention Time of TIC Peaks Unique to MTb

| | mean retention (min) | Sd | % rsd |
|---|---|---|---|
| Peak 1 | 4.433 | 0.007 | 0.17 |
| Peak 2 | 4.643 | 0.009 | 0.19 |
| Peak 3 | 5.70 | 0.01 | 0.19 |
| Peak 4 | 5.80 | 0.01 | 0.20 |
| Peak 5 | 8.90 | 0.01 | 0.15 |
| Peak 6 | 12.50 | 0.02 | 0.17 |

Six of the seven chromatographic peaks were identified based on chromatographic figures of merit, mass spectra evaluation, and comparison to the NIST library. Subsequent sections provide the detailed analysis of the chromatographic and mass spectral data, however Table D summarizes the total ion abundances and peak identities based on matches to the NIST library. Briefly, the NIST library search mechanism employs three approaches to compound identification: forward search, reverse search, and match probability. Both forward and reverse searches are based on an ideal score of 1000 in the event of a perfect match between an unknown spectrum and a library spectrum. Benchmark parameters used in practice are values ≧900 are an excellent match, values between 800 and 900 are good, values between 700 and 800 are fair, and anything under 600 is poor. In a forward search, the software compares the unknown mass spectrum to the spectra of known compounds, and the resulting score reflects how closely the unknown spectrum matches the known compounds' mass spectra. Extra peaks in the unknown spectrum that are absent in the known spectrum result in a lower score for the match between the two compounds. Reverse searches, by contrast, attempt to find known compounds in the mass spectrum of the unknown and assumes that a peak present in the unknown but absent in the library spectrum is an impurity.

TABLE D
Peak^{a} Abundances and Library Matches for Unique MTb Peaks in TICs

| | | File | | | | | | | |
|---|---|---|---|---|---|---|---|---|---|
| | Peak | 13_4 | 13_6 | 13_9 | 13_10 | 14_4 | 14_6 | 15_5 | 15_7 |
| methyl propionate | 1 | 8600 | 5600 | 10000 | 10000 | 11000 | 11000 | 6000 | 5600 |
| 2-butanone | 2 | 10000 | 6000 | 10000 | 10000 | 10000 | 10000 | 8000 | 6000 |
| 3-pentanone | 3 | 8000 | 5200 | 8000 | 8000 | 7000 | 8000 | 5000 | 4400 |
| 2-pentanone | 4 | 4200 | 3200 | 4000 | 4000 | 5000 | 5000 | 2000 | 2000 |
| methoxybenzene | 5 | 4000 | 3500 | 4000 | 3900 | 3500 | 4000 | 1600 | 1800 |
| *see below | 6 | 4500 | 3300 | 4000 | 3900 | 3600 | 3500 | 1900 | 2100 |

^{a}= Peaks are arranged by increasing retention time. See Table C.
*= Peak 6 is 2-ethylhexanoic acid methyl ester.

Match probability values are reported as a percent, and this score expresses the likelihood that the unknown spectrum will be correctly matched to a known compound in the database. As such, an unknown compound whose mass spectrum may be matched to a multitude of database entries will receive a lower score than an unknown compound with a highly-unique ion profile. Accordingly, a given mass spectrum may merit favorable scores in the forward and reverse searches but still earn a low score in the match probability category. Interpretation of the probability value parallels that of the forward and reverse searches but is based on an ideal score of 100. Finally, it is understood that there may be considerable variability in score assignments using the NIST library, which often has multiple entries for the same compound. The main library is the entry judged to be the best spectrum by NIST personnel, however the replicate library contains additional entries submitted by others. As such, when scores are provided for a given mass spectrum, different replicates may be compared to either the main library mass spectrum or to any library replicates provided. In the analyses provided here, no distinction is made between the reference mass spectra (i.e., main or replicate library entry) that are being matched. It is assumed that no sensible error is introduced in the spectral interpretation, although there is some variability among the library entries.

An understanding of the various NIST scores for a given chromatographic peak is relevant, as several external factors may have deleterious effects on the resulting score a mass spectrum earns. Typically, peaks with low total ion abundances tend to earn lower scores on the basis of poor signal to noise ratios. If the chromatographic peak is not particularly large (i.e., 5000 or greater for total ion abundance), the purity of the peak may be somewhat suspect and the mass spectrum may not include all ion fragment peaks at a measurable intensity. In addition, it was noted in the evaluation of the data that all mass spectra for MTb files 13_{—}4, 6, 9, 10 and 14_{—}4, 6 contained exceptionally large peaks at m/z values of 40 and 44. In some cases, these peaks had the greatest absolute abundance in the mass spectrum and so were weighted heavily in the comparison to the NIST library reference spectra. These peaks are thought to arise from argon (m/z=40) and carbon dioxide (m/z=44) based on their prevalence throughout every mass spectrum, regardless of where in the chromatogram it was selected. The source of these two compounds should be external to the sample, as they elute almost at the same time as the void time (the time required for a nonretained solute to elute from the column) of the instrumental system. Because these peaks appear in every spectrum and therefore must have a continuous source, they clearly were not originating from the SPME fibers. In all likelihood, the source of the argon is the helium carrier gas and the carbon dioxide probably was introduced from an air leak in the mass-selective detector. Regardless, the constant presence of these two peaks in the six files served to decrease the quality of the matches against the NIST library. Accordingly, the effect of these peaks was considered in the results described below.

To validate the identity of various chromatographic peaks, known standards may be run via the same methods and under the same conditions, to the extent possible, as used in the analysis of MTb. The retention time of a standard compound should not be significantly different from that of the MTb chromatographic peak and should produce a mass spectrum that closely matches that of the library. In addition, the mass spectrum of the standard taken in this way may be compared to the mass spectra of the unknown compounds to confirm the integrity of peak identification. This validation approach allows for accurate peak identifications, except in the rare circumstance where two analytes coelute from the column. To this end, mass spectra of the MTb samples may be viewed over different regions of the chromatographic peak to determine if the peaks were not chromatographically pure. In the analysis described below, no evidence of coelution was found.

2b. Detection of Methyl Propionate as a Compound Indicative of MTb in a Sample.

Peak 1 MTb replicates were initially identified using the NIST library, and unique match values were returned for methyl propionate in every case. No other library spectrum meaningfully matched the mass spectrum of the compound, and the search results are summarized in Table E. The NIST library contains a total of five mass spectrum entries for methyl propionate (the main library plus four replicates).

TABLE E
NIST Library Matches for MTb Peak 1 (tᵣ = 4.433 ± 0.007)
with Methyl Propionate

| File | Forward | Reverse | Probability |
|---|---|---|---|
| 13_4 | 731 | 759 | 68.1 |
| 13_6 | 660 | 707 | 61.4 |
| 13_9 | 779 | 793 | 79.3 |
| 13_10 | 773 | 803 | 80.1 |
| 14_4 | 766 | 815 | 80.8 |
| 14_6 | 752 | 800 | 75.5 |
| 15_5 | 828 | 862 | 83.2 |
| 15_7 | 830 | 866 | 89.3 |

All forward and reverse search scores were fair to good, with the exception of the forward search for file 13_{—}6. The reverse search scores were consistently higher than the forward scores. Even more encouraging were the probability match scores, where six of the eight files match better than 70%, the threshold value for determining the uniqueness of a compound's mass spectrum relative to the library. These results indicated that Peak 1 is methyl propionate.

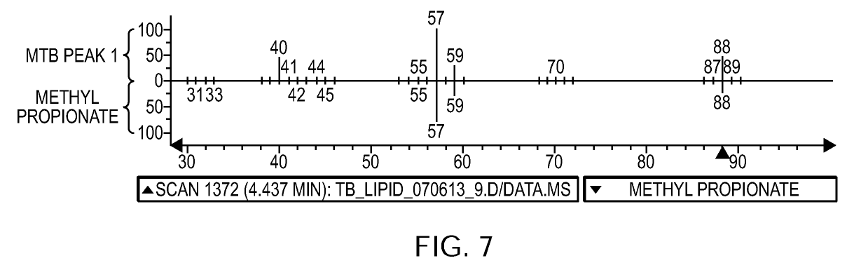

The individual MTb replicate mass spectra were compared to the library spectra, and of particular interest are the relative peak abundances for the characteristic m/z peaks. Since there are five reference spectra for methyl propionate in the NIST library, there is some variability in the relative peak abundances. Moreover, most of the reference spectra have 5-6 peaks whose m/z values are less than 39; the instrumental method used in this experiment has no m/z values less than 39. This impacts peak scoring, because characteristic peaks are absent in the MTb spectra. Using the NIST reference spectra, the expected m/z values are, in decreasing order of relative abundance: 57>88≈59>45≈55. As the principle peak, the relative abundance of m/z=57 is 100%, while the peaks at 88 and 59 are approximately 20-35% and the remaining two peaks are about 4-6% relative abundance. As such, only a limited comparison of the relative abundance values were made. A representative head-to-tail comparison of the MTb and methyl propionate reference spectra are shown in FIG. 7, with a detailed comparison of relative peak intensities provided in Table F.

TABLE F
Relative Ion Abundances for Methyl Propionate in MTb Replicates.

| | m/z | | | | | | | | | |
|---|---|---|---|---|---|---|---|---|---|---|
| | 57 | 88 | 59 | 45 | 55 | 43 | 42 | 56 | 44 | 40 |
| 13_4 | 100 | 33.0 | 33.4 | 6.3 | 6.9 | 7.8 | 4.2 | 4.6 | 20.5* | 57.8* |
| 13_6 | 100 | 39.7 | 39.0 | 6.3 | 7.6 | 5.9 | 7.4 | 6.4 | 32.4* | 113* |
| 13_9 | 100 | 36.6 | 39.8 | 5.6 | 3.8 | 5.4 | 3.3 | 2.9 | 12.2* | 42.8* |
| 13_10 | 100 | 35.9 | 36.6 | 5.4 | 6.7 | 8.4 | 3.9 | 2.4 | 12.3* | 45.2* |
| 14_4 | 100 | 37.7 | 26.4 | 3.9 | 6.5 | 4.9 | 4.2 | 3.8 | 15.7* | 43.8* |
| 14_6 | 100 | 36.1 | 29.8 | 5.2 | 4.3 | 4.0 | 3.6 | 4.1 | 15.1* | 56.6* |
| 15_5 | 100 | 35.4 | 32.2 | 4.3 | 6.5 | 7.2 | 2.2 | 4.7 | 10.4* | 2.6 |
| 15_7 | 100 | 43.5 | 30.5 | 6.1 | 7.0 | 7.4 | 3.6 | 2.5 | 10.7* | 4.1 |

*= Ions indicated by asterisks are suspected of arising from argon (m/z = 40) and carbon dioxide (m/z = 44). Thus, abundances of these ions likely are unrelated to compound identity.

Examination of FIG. 7 illustrates that additional peaks at m/z values <30 are visible in the reference spectrum. Small peaks not present in the reference spectrum appear throughout the mass spectrum of the MTb sample. As indicated previously, the relative ion abundances for argon and carbon dioxide are quite substantial for most MTb files (indicated with an asterisk in Table F), and this further obscures the quality of the match. If these values are disregarded as systematic errors, then it is clear that the relative ion abundances match reasonably well to those of the reference mass spectra. The m/z=57 peak is the most abundant in all cases, the peaks at 59 and 88 generally range from 20-35% relative abundance, while those of m/z=45 and 55 are markedly lower (all less than 8%). Collectively, these data indicate that methyl propionate is the identity of the compound.

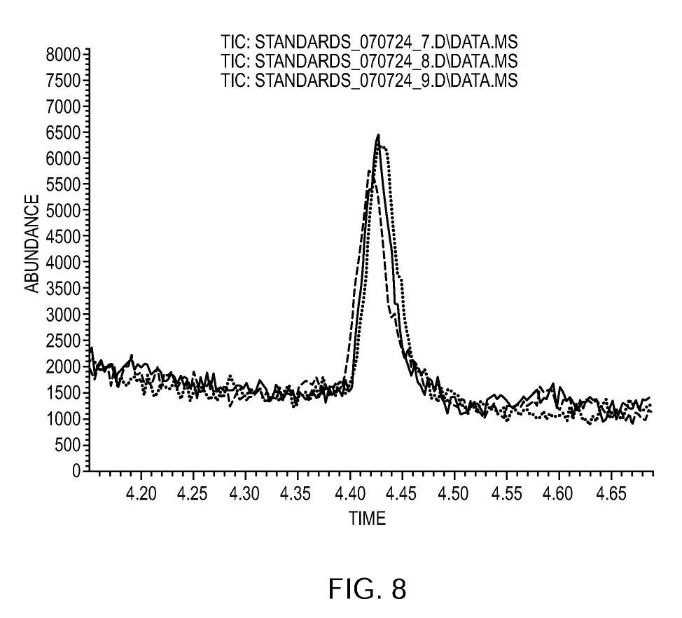

This was corroborated using known methyl propionate. A solution of approximately 20 ppmv methyl propionate (Aldrich, 99%) in ethanol (Sigma-Aldrich, 200 proof) was prepared so that the resulting total ion abundance in the TIC would be comparable to that observed in the MTb files. The headspace of the solution was extracted for five minutes using SPME fibers, and the experiment was performed in triplicate. The relevant data files are 24_{—}7, 8, and 9, and the relevant portion of the TICs is shown in FIG. 8. The average retention time for methyl propionate was found to be 4.427±0.004 (% rsd=0.10%), which compares favorably to that of Peak 1 in the MTb results (4.433±0.007). A t-test performed at the 95% confidence level on the two averages shows no statistically significant difference between the two retention times, which further indicates the identification of Peak 1 as methyl propionate.

The mass spectra of the standards were compared to those of the NIST spectral library, and the search results are listed in Table G. The mass spectra of the standard methyl propionate solutions all yielded good forward and reverse searches, and the match probability values were excellent.

TABLE G
NIST Library Matches for Methyl Propionate Standard

| File | Forward | Reverse | Probability |
|---|---|---|---|
| 24_7 | 866 | 897 | 91.0 |
| 24_8 | 843 | 899 | 90.7 |
| 24_9 | 841 | 882 | 87.8 |

The relative ion abundances of the standards also confirm the mass spectra of both the NIST library and the MTb peaks. Specifically, the m/z=57 peak had a 100% relative abundance value, while the m/z peaks at 88 and 59 were comparable and ranged from 30-43%. The m/z=45 relative peak abundance for the methyl propionate standards was notably higher than those of the MTb samples (roughly 25% vs. 5%), however all remaining peaks, excluding m/z=40 and 44, listed in Table F had relative abundance values comparable to those of the MTb samples. Collectively, these data unambiguously indicate that the identity of Peak 1 is methyl propionate.

2c. Detection of 2-Butanone as a Compound Indicative of MTb in a Sample.

Peak 2 replicates were evaluated against the NIST library, and five of the eight replicates best matched 2-butanone. The remaining peaks were mostly matched to 6,10-dimethyl-(E,E)-5,9-dodecadien-2-one (C₁₄H₂₄O). The results of the library searches are shown in Table H. The NIST library had four total entries for 2-butanone: the main library mass spectrum as well as three replicates.

TABLE H
NIST Library Matches for MTb Peak 2 (tᵣ = 4.643 ± 0.009)

| | | | | 6,10-dimethyl-(E,E)- | | |
|---|---|---|---|---|---|---|
| | | | | 5,9-dodecadien-2-one | | |
| | 2-butanone | | | | Re- | |
| File | Forward | Reverse | Probability | Forward | verse | Probability |
| 13_4 | 773 | 797 | 37.1 | 776 | 804 | 42.0 |
| 13_6 | 597 | 819 | 2.94 | 691 | 731 | 64.7 |
| 13_9 | 779 | 911 | 62.7 | 750 | 774 | 17.8 |
| 13_10 | 790 | 909 | 63.9 | 762 | 781 | 18.4 |
| 14_4 | 738 | 782 | 37.2 | 733 | 773 | 30.0 |
| 14_6 | 712 | 742 | 13.4 | 759 | 787 | 64.1 |
| 15_5 | 836 | 866 | 69.6 | 766 | 789 | 6.08 |
| 15_7 | 832 | 855 | 50.6 | 757 | 775 | 5.49 |

Examination of Table H of the forward and reverse search results for 2-butanone shows that in all but one case, the search matches range from fair to excellent. Only one forward search result is poor. The forward and reverse search results for C₁₄H₂₄O are all effectively good. For both compounds, the reverse search always earns a higher score than the forward search, which is expected since the forward search penalizes for any additional peaks appearing in the unknown that are absent in the known spectrum. Regardless of which library entry is used for 2-butanone, the results are the same. The probability match for both compounds did not exceed the desired threshold value of 70% in any replicate.

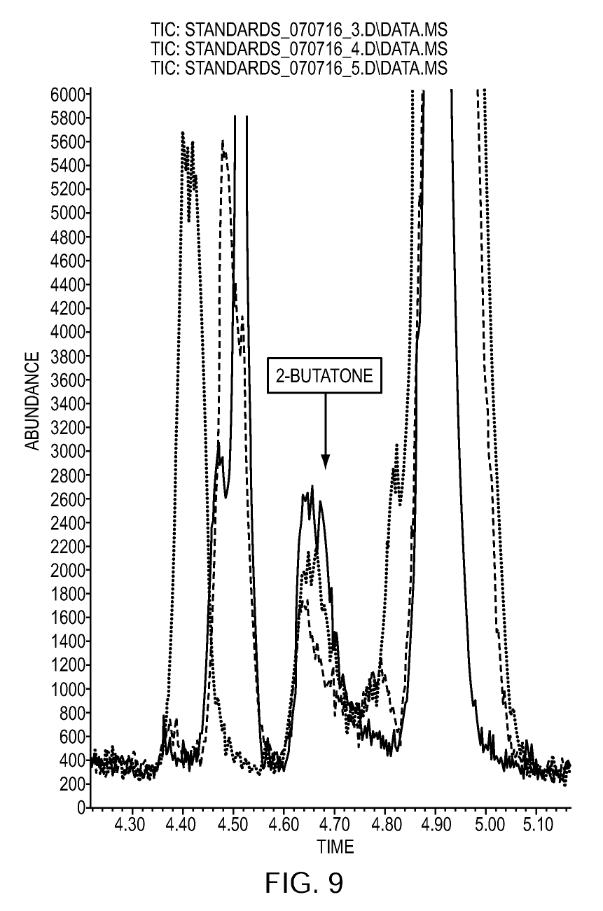

However, to identify the MTb Peak 2, an approximately 20-ppmv 2-butanone (Sigma-Aldrich, 99+%) standard solution was prepared in methanol (Sigma, M1770-1L). This concentration was chosen to yield a total ion abundance comparable to that obtained for the MTb files. The resulting solution headspace was extracted in triplicate for five minutes using SPME fibers. After extraction, the samples were analyzed using the dual-detector GC-MS system with trap-and-purge, and the relevant data files are 16_{—}3, 4, and 5. The resulting TICs are shown in FIG. 9 and the average retention time for 2-butanone was 4.652±0.009 (rsd=0.20%). Comparison of the 2-butanone retention time with that of MTb Peak 2 showed no significant difference in retention time at the 95% confidence level. This result indicates that the MTb Peak 2 is 2-butanone.

The standard 2-butanone mass spectra were then compared to the NIST spectral library entries for both 2-butanone and C₁₄H₂₄O and the results are shown in Table I.

TABLE I
NIST Library Matches for 2-Butanone Standard

| | | | | 6,10-dimethyl-(E,E)- | | |
|---|---|---|---|---|---|---|
| | 2-butanone | | | 5,9-dodecadien-2-one | | |
| File | Forward | Reverse | Probability | Forward | Reverse | Probability |
| 16_3 | 770 | 874 | 67.2 | 734 | 779 | 16.6 |
| 16_4 | 776 | 889 | 69.0 | 746 | 771 | 19.2 |
| 16_5 | 732 | 872 | 62.3 | 684 | 734 | 6.93 |

As was observed with the MTb replicates, the forward and reverse searches for the 2-butanone standard were generally good to fair matches. Although the probability matches did not reach the threshold of 70%, the matches show improvement in spectral uniqueness over the MTb replicates for 2-butanone, and the matches to C₁₄H₂₄O were poor. Similarly, the forward and reverse searches for the standard and C₁₄H₂₄O were all fair, with one exception in file 16_{—}5. The reverse search always returned a higher score than the forward search. Collectively, these results indicate that the NIST library spectral match may not predict the minimum desired probability of a match between 2-butanone with the NIST library. No doubt, the low total ion abundance of both the standard (˜1600-2800) and the MTb replicates (6000-10000) observed in the TICs contributes to a lower-quality match. However, the probability of a good match is decidedly worse for C₁₄H₂₄O for both the 2-butanone standard and the MTb replicates.

To aid in the identification of the unknown compound, the relative abundances of m/z values in the various library entries of the known compounds were compared. These results are summarized in Table J.

TABLE J
Relative Ion Abundances for NIST Library Entries.^{a}

| | m/z | | | | | | | | | |
|---|---|---|---|---|---|---|---|---|---|---|
| | 43 | 72 | 57 | 42 | 44 | 58 | 55 | 41 | 71 | 85 |
| Main^{b} | 100 | 25 | 8 | 3 | 3 | | | | | |
| Rep 1^{b} | 100 | 19.9 | 6.6 | 4.3 | 2.5 | | | | | |
| Rep 2^{b} | 100 | 22 | 7.9 | 4 | 2.5 | | | | | |
| Rep 3^{b} | 100 | 24.3 | 7 | 4.1 | 2.4 | | | | | |
| Main^{c} | 100 | 37.7 | 12.7 | 7.8 | 4.8 | 6.2 | 3.7 | 3.5 | 3.4 | 3.3 |

^{a}= Ion m/z values are listed in descending order of relative abundance based on 2-butanone.
^{b}= NIST reference spectra for 2-butanone. The main library source is C. Djerassi, Replicate 1 is Chemical Concepts, Replicate 2 is Japan AIST/NIMC Database, and Replicate 3 is Dow Chemical Company.
^{c}= NIST reference spectrum for 6,10-dimethyl-(E,E)-5,9-dodecadien-2-one (C₁₄H₂₄O). The main library source is the Insect Chem. Ecol. Lab

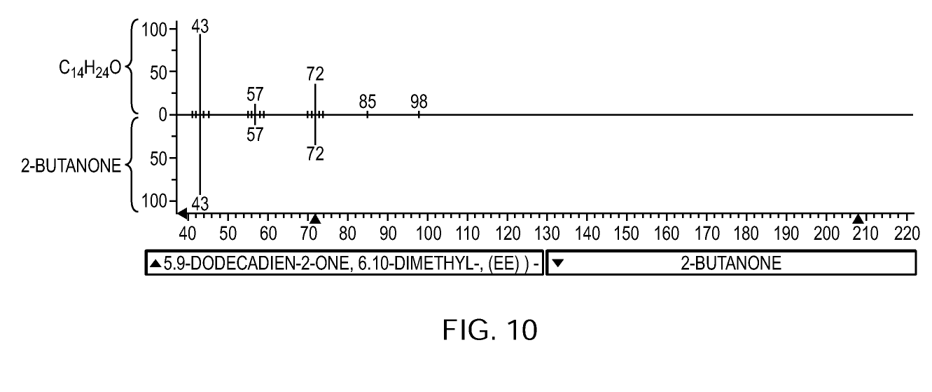

As indicated in Table J, it is clear that each of the four 2-butanone library mass spectra have the same sequence of relative abundance values: 43>72>57>42>44. The relative abundances are fairly comparable between the four spectra. An almost identical sequence, with comparable relative abundances, is observed for the C₁₄H₂₄O spectrum; only m/z=44 is out of sequence, since m/z=58 has a slightly higher relative abundance. These data indicate that the 2-butanone mass spectrum is essentially a subset of the main ion peaks of C₁₄H₂₄O and this is depicted graphically in FIG. 10 via the head-to-tail comparison of the two library spectra. This phenomenon is partly responsible for the poor probability match values for the MTb peaks, since the mass spectra themselves are not substantially unique.

The relative abundances of the principle ions for the 2-butanone standard and MTb replicates were then assessed. The anomalously large peaks at m/z=40 and 44 for six of the eight replicates introduced some error into the spectral interpretation of, and comparison to, the NIST library. With this in mind, the relative abundances of the principle ions were calculated for all MTb and 2-butanone standard replicates without inclusion of these two peaks. The resulting values are shown in Table K. In Table K, the relative ion abundances largely matched well with both the 2-butanone and C₁₄H₂₄O NIST library spectra (see Table J). Nevertheless, the comparability of the relative ion abundances added to the evidence that Peak 2 is either 2-butanone or C₁₄H₂₄O.

TABLE K
Relative Ion Abundances for 2-Butanone and MTb Replicates.

| m/z^{a} | 43 | 72 | 57 | 42 |
|---|---|---|---|---|
| 13_4^{b} | 100 | 29.8 | 9.3 | 5.9 |
| 13_6^{b} | 100 | 21.3 | 7.3 | 6.8 |
| 13_9^{b} | 100 | 27.9 | 8.0 | 6.5 |
| 13_10^{b} | 100 | 26.7 | 8.3 | 7.1 |
| 14_4^{b} | 100 | 21.4 | 8.8 | 5.6 |
| 14_6^{b} | 100 | 25.9 | 8.0 | 7.4 |
| 15_5^{b} | 100 | 30.0 | 9.3 | 6.0 |
| 15_7^{b} | 100 | 27.8 | 7.5 | 6.9 |
| 16_3^{c} | 100 | 39.6 | 12.2 | 8.8 |
| 16_4^{c} | 100 | 29.1 | 8.2 | 4.3 |
| 16_5^{c} | 100 | 29.5 | 7.8 | 10.3 |

^{a}= Ion m/z values listed in descending order of relative abundance as determined from Table J.
^{b}= MTb replicates.
^{c}= 2-butanone standards.

The chromatographic evidence points to the identity of Peak 2 as 2-butanone. Moreover, it is unlikely that C₁₄H₂₄O could coelute with 2-butanone. The formula weight of C₁₄H₂₄O is 208 while that of 2-butanone is 72. While the boiling point of C₁₄H₂₄O is unknown, it is unrealistic to think that it is identical to that of 2-butanone and/or that it would undergo identical retention characteristics in the chromatographic column. Nevertheless, it is instructive to look at other MS features that may contribute to less than ideal matches.

If the unknown compound were C₁₄H₂₄O, additional peaks would be expected at m/z=58, 55, 41 and 71. Of particular interest would be the presence of m/z values greater than 72, since this is the molecular weight of 2-butanone, and no peaks greater than that are observed in 2-butanone spectra. For C₁₄H₂₄O, the peaks of note, in order of relative abundance, are: 85, 83, 84, and 98. The relative abundances of these peaks are on the order of 1-3%. Looking at the mass spectra of the 2-butanone standards, none of these peaks is observed except for a small peak in file 16_{—}4 at m/z=85 with an absolute abundance of 12. In the MTb files, files 13_{—}4 and 14_{—}6 each have small peaks at m/z=85 with absolute abundances of 11 and 17, respectively. These values correspond to relative abundances between 0.25-1.0%, with 1.0% relative abundance being calculated for the standard 2-butanone, which does not contain C₁₄H₂₄O. In sum, there is no meaningful evidence in the mass spectra to support the identification of Peak 2 as C₁₄H₂₄O. While it would be most convincing to inject a standard of this compound, no CAS number is provided for the compound in the NIST library, and it is unlikely to be commercially available. As such, the above-identified experiments indicate that Peak 2 is 2-butanone.

The final area of concern in the mass spectral data is the presence a small peak around 4.6 min in the lipid media TICs, however the total abundance of the peak was not substantial. Overall, the average retention time for the peak in the media was 4.644±0.007 (% rsd=0.16%) which is not significantly different at the 95% confidence level from the mean of the 2-butanone peak in the MTb samples. These data suggest that the media peak is also 2-butanone. The range of absolute ion abundance values seen in the media peaks was 1200-5500, with the peak at 5500 anomalously larger due to a high chromatographic background signal. By contrast, this peak in the MTb samples ranged from 5700-10200. As such, the highest media peak has a lower abundance than the least abundant MTb peak. Overall, the average peak abundance for the media is 3400±1300 while that of the MTb peaks is 8600±1900. The two averages are significantly different at the 95% confidence level, so it may be concluded that the MTb samples contain more 2-butanone than the media alone.

The mass spectra of the media peaks were then evaluated to confirm their identity, and the results are shown in Table L. All forward matches with 2-butanone scored poorly, as did half of the reverse searches. The match probability also scored poorly for 2-butanone, and the media peaks scored favorably to 6,10-dimethyl-(E,E)-5,9-dodecadien-2-one in all categories. Evaluation of the mass spectra reveals that the primary problem with the peak misidentification is the presence of spurious peaks in the media's mass spectra. These include the peaks at m/z=40 and 44 in the 13_and the 14_files in addition to other anomalies from additional sources (the column, SPME, etc.). Since the mass spectral peaks are not intense, these spurious peaks weigh disproportionately in the mass spectrum and decrease the quality of the match. These data indicate that while 2-butanone does appear in the lipid media, there is reproducibly more of this compound in the MTb.

TABLE L
Spectral Matches for Lipid Media

| | for- | | | | forward/rev/ |
|---|---|---|---|---|---|
| file | ward^{a} | reverse^{a} | probability^{a} | best match | probability |
| 13_3 | 516 | 560 | 2.27 | C₁₄H₂₄O^{c} | 606/679/44.3 |
| 13_4 | 475 | 729 | 0.12 | C₁₄H₂₄O | 626/694/37.8 |
| 13_11 | 550 | 813 | 1.41 | C₁₄H₂₄O | 648/697/55.2 |
| 13_12 | 552 | 805 | 2.13 | C₁₄H₂₄O | 665/742/59.3 |
| 14_3 | 551 | 663 | 0.31 | C₁₄H₂₄O | 677/743/38.3 |
| 14_5 | 540 | 605 | 1.14 | C₁₄H₂₄O | 659/743/50.5 |
| 15_4^{b} | n/a | n/a | n/a | 2,3- | 600/762/21.6 |
| | | | | dimethyloxirane | |
| 15_6 | 639 | 802 | 18.7 | C₁₄H₂₄O | 649/725/26.4 |

^{a}= Spectral match values are for 2-butanone.
^{b}= No listing for 2-butanone appeared in the top 100 matches for this peak.
^{c}= This compound is 6,10-dimethyl-(E,E)-5,9-dodecadien-2-one.

2d. Detection of 3-pentanone as a Compound Indicative of MTb in a Sample.

MTb Peak 3 yielded a favorable comparison to the NIST library for 3-pentanone. The library contains four total entries for this compound: the main library plus three replicates, and the match results are summarized in Table M.

TABLE M
NIST Library Matches for MTb Peak 3 (tᵣ = 5.67₂ ± 0.01₁)
with 3-Pentanone

| File | Forward | Reverse | Probability |
|---|---|---|---|
| 13_4 | 697 | 744 | 55.8 |
| 13_6 | 668 | 767 | 63.9 |
| 13_9 | 710 | 758 | 60.5 |
| 13_10 | 733 | 777 | 64.7 |
| 14_4 | 698 | 803 | 60.7 |
| 14_6 | 719 | 810 | 56.6 |
| 15_5 | 813 | 911 | 77.8 |
| 15_7 | 778 | 898 | 66.6 |

Table M shows that seven of the mass spectra yielded fair to good forward matches for 3-pentanone, while all reverse spectral matches were fair to excellent. As usual, the reverse match scores are higher than the forward match scores due to the presence of spurious peaks in the mass spectra. The probability matches for uniqueness were mostly poor (i.e., <70%), however five of the eight were in the 60-70% range and one was almost 80%. As such, these results indicate that identity of Peak 3 is 3-pentanone.

The mass spectral relative abundances were then compared to the NIST library spectra, however the four library entries have varying spectra. All four spectra further have four of 10 of the most intense peaks occurring at m/z values <39, which is the minimal ion value measured in the MTb mass spectra. Undoubtedly, this contributes to the low-quality matches, since these are spectral characteristics used to determine compound identity in the match algorithm. Moreover, both the MTb and NIST mass spectra have two large peaks at m/z=57 and 86, with seven additional peaks whose relative abundances are very comparable (i.e., 1.5-6.5%). Of these seven peaks, only one peak, m/z=58, appears in all four standard spectra in the top-ten highest relative abundances. Finally, the MTb mass spectra for files 13_{—}4, 6, 9, 10 and 14_{—}4, 6 have exceptionally large peaks at m/z=40 (argon) and 44 (carbon dioxide). The argon peaks for these files range in relative abundance values from 47-89%, while those of files 15_{—}5, 7 are only ˜2.5%. Carbon dioxide peaks for the files obtained on the 13_and 14_files range from 14-22% while those for the 15^{th} were 6-8%. Large, spurious peaks produce lower scores and probability matches regardless of which strategy is used.

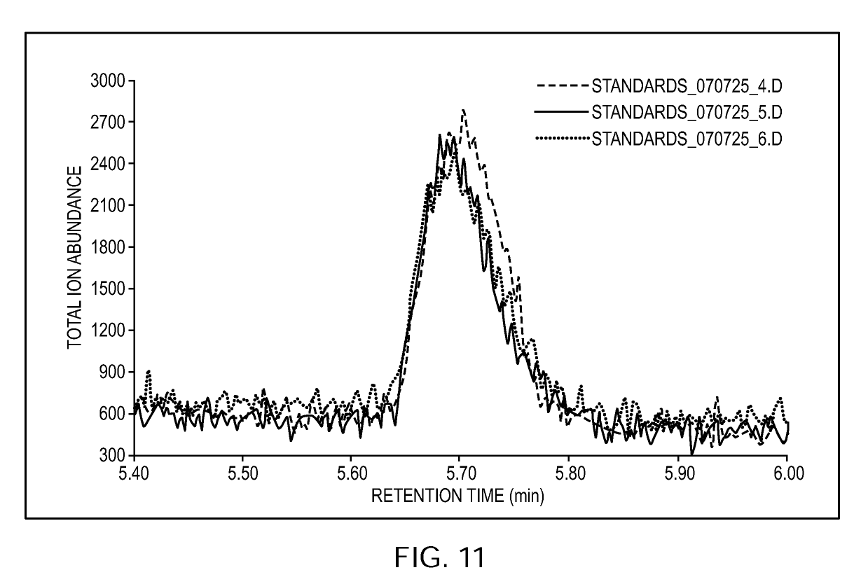

To confirm the identity of Peak 3 as 3-pentanone, a solution of roughly 30 ppmv 3-pentanone (Sigma-Aldrich, ReagentPlus, ≧99%) was prepared in ethanol (Sigma-Aldrich, 200 proof). This concentration was chosen so that the total ion abundance of the standard's chromatographic peak roughly matched that measured in the MTb samples. The solution headspace was extracted in triplicate using SPME fibers and was subsequently analyzed using the dual-detector GC-MS system with trap-and-purge. The relevant data files are 25_{—}4, 5, and 6, and the relevant portion of the TICs is shown in FIG. 11. The average retention time for the 3-pentanone standards was 5.696±0.006 (% rsd=0.11%) which appears to be in good agreement with the average retention time of the MTb files (5.67₂±0.01₁). An F-test was performed on the standard deviations of the two means, and they are not significantly different at the 95% confidence level. The average retention times for the standard and the MTb files are, however, different at the 95% confidence level based on t-test results.

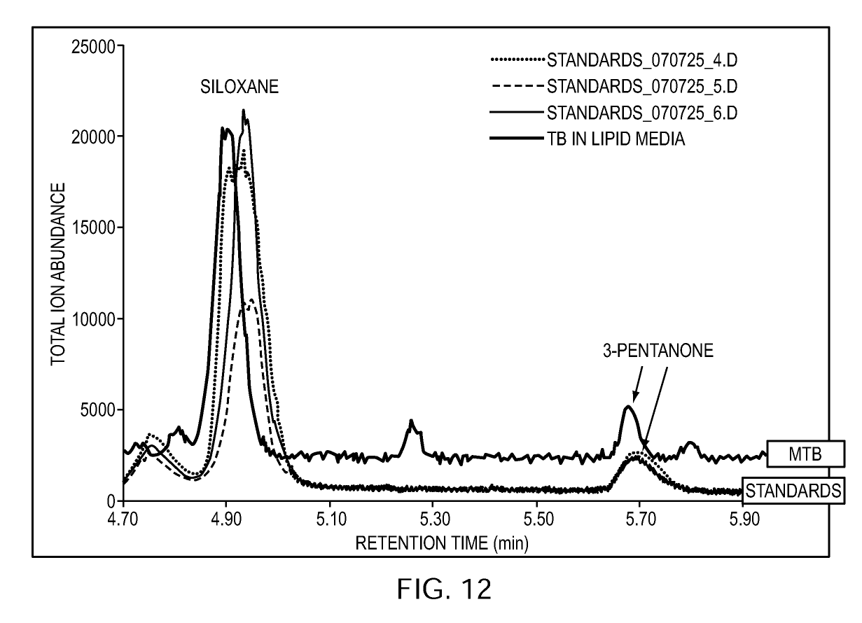

In order to understand the difference in retention times, representative TICs of MTb and the 3-pentanone standards were overlaid and are shown in FIG. 12. It is clear from FIG. 12 that the MTb 3-pentanone peak is narrower and has a greater total ion abundance than that of the standard. Peak shape (i.e., height versus width and peak symmetry) may affect the overall retention time of a compound as the center of mass for the peak shifts. The 3-pentanone peaks in the standards and MTb files have about a 1.4-second difference in average retention time, which could easily be caused by instrumental factors such as a small change in flow rate. If this were the case, it is reasonable to expect a systematic offset in other chromatographic peaks by roughly the same time differential. Because the 3-pentanone is a reasonably pure standard, there are not many additional peaks in the chromatogram to compare. (The ethanol peak cannot be used, since this peak is disproportionately large in the 3-pentanone chromatogram.) The siloxane peak at about 4.9 minutes is the only reasonable peak for comparison and this is labeled in FIG. 12.

In FIG. 12, it is clear that the siloxane peak in the MTb file has a shorter retention time (average for eight MTb replicates: 4.90₈ min±0.01₁) than the retention time of the siloxane peaks (average for three standard replicates: 4.93₃ min±0.01₀) in the 3-pentanone standard chromatograms. The two means are significantly different at the 95% confidence level, and the time difference between the two averages is roughly 1.5 seconds. This result is clearly comparable to the time difference observed in the 3-pentanone peaks, thus suggesting that the difference is systematic, hence instrumental, in origin. Overall, the difference in retention time is fairly small, so that the retention time of 3-pentanone indicates that the identification of MTb Peak 3 is 3-pentanone.

Next the mass spectra of the library, 3-pentanone standards, and MTb replicates were compared, and these results are shown in Table N. The principle peak in all cases was m/z=57, with the parent ion peak (i.e., the m/z value for the +1 ion of 3-pentanone with no fragmentation) at m/z=86 the next most abundant ion. Interestingly, no peak was observed at m/z=45 for the four library entries, and this peak was barely measurable in six of the eight MTb replicate mass spectra. The 3-pentanone standards, however, each had peaks at this value of roughly 11%. It is unclear what the source of this fragment in the standards would be, since it is essentially immeasurable in the NIST standard libraries. Specifically, Replicates 2 and 3 have no measurable peak in their reference spectra, and a trivial peak is observed in the spectra for the Main and Replicate 1 library entries. In all cases, the peak doesn't exceed more than 1% relative abundance. As such, this peak in the standards may be an impurity, and it is not observed in any of the standard or MTb entries. All other remaining peaks appear at reasonably comparable relative intensity values, with the exception of no peak at m/z=39 for MTb files 13_{—}6 and 14_{—}6. This is consistent with the observation that this peak is the lowest m/z value measured by the mass spectrometer and sometimes does not appear. Moreover, the peak is quite small and is absent in three of the four library replicates. Accordingly, the mass spectral data supports the identity of Peak 3 as 3-pentanone.

TABLE N
Relative Ion Abundances for 3-Pentanone Mass Spectra in the
NIST Library, Standards, and MTb Files

| | m/z | | | | | | | | |
|---|---|---|---|---|---|---|---|---|---|
| | 57 | 86 | 45 | 56 | 58 | 41 | 42 | 43 | 39 |
| Main | 100 | 21.1 | NR | 3.6 | 3.3 | 1.9 | 1.4 | NR | NR |
| Rep 1 | 100 | 17.3 | NR | 3.5 | 3.5 | NR | 3.9 | 4.2 | NR |
| Rep 2 | 100 | 22.5 | NR | NR | 3.2 | NR | 4.3 | 2.7 | 2.7 |
| Rep 3 | 100 | 23.9 | NR | 7.1 | 5.9 | 1.9 | NR | 2.8 | NR |
| 25_4 | 100 | 24.2 | 11.1 | 4.5 | 4.0 | 1.9 | 4.8 | 5.0 | 4.0 |
| 25_5 | 100 | 20.6 | 11.4 | 4.3 | 5.2 | 3.6 | 3.3 | 4.2 | 2.5 |
| 25_6 | 100 | 20.3 | 10.3 | 2.6 | 3.0 | 3.5 | 3.8 | 5.1 | 2.6 |
| 13_4 | 100 | 25.4 | 1.3 | 3.6 | 5.7 | 3.2 | 3.3 | 3.0 | 2.0 |
| 13_6 | 100 | 27.5 | NM | 6.5 | 2.6 | 4.2 | 3.7 | 2.6 | NM |
| 13_9 | 100 | 29.0 | 0.5 | 5.7 | 4.7 | 2.4 | 3.4 | 3.8 | 2.7 |
| 13_10 | 100 | 23.5 | 0.7 | 4.8 | 5.1 | 3.1 | 3.8 | 3.0 | 3.4 |
| 14_4 | 100 | 22.1 | 0.3 | 4.4 | 3.3 | 3.6 | 3.6 | 2.9 | 1.6 |
| 14_6 | 100 | 24.0 | 0.3 | 5.0 | 1.7 | 3.0 | 2.3 | 3.8 | NM |
| 15_5 | 100 | 20.5 | 0.4 | 2.8 | 4.2 | 1.4 | 2.8 | 3.6 | 2.3 |
| 15_7 | 100 | 27 | NM | 5.0 | 3.5 | 3.7 | 2.1 | 2.8 | 3.4 |

NR = not reported for the standards; only the fragments with the top-ten largest relative abundance values are reported in the library
NM = not measured for the MTb replicates; this means no peak appeared at the m/z value

2e. Detection of 2-pentanone as a Compound Indicative of MTb in a Sample.

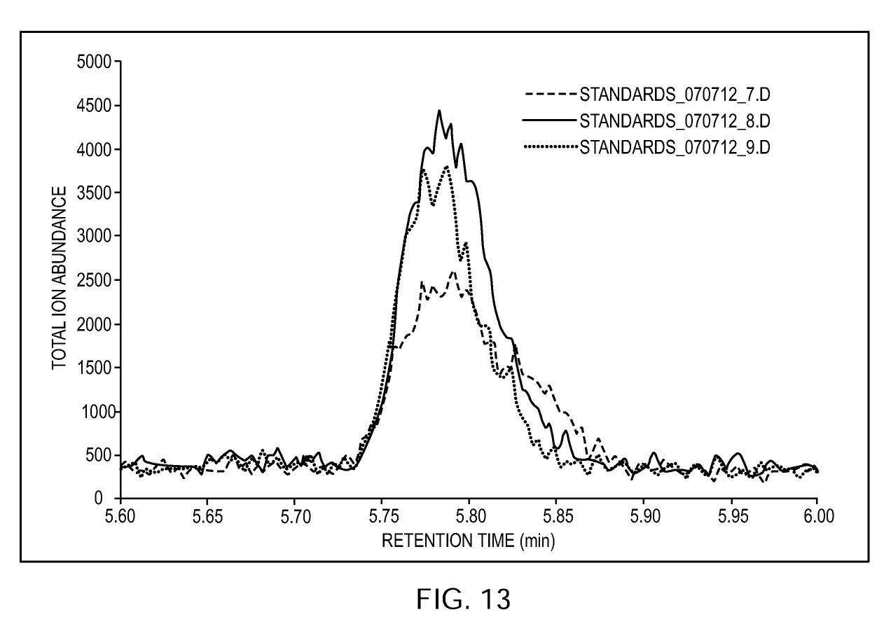

Comparison of the MTb mass spectra to the NIST library did not yield a strong (i.e., ≧70%) probability match for 2-pentanone. Additionally, the forward and reverse search results generally produced poor matches with the NIST library spectra. The mass spectra for the eight replicates generally yielded a better probability match for 2-methyl-N,N-diisopropyl-propanamide. To determine the peak identity, a standard solution of about 20 ppmv 2-pentanone (Aldrich, HPLC grade) in methanol (Sigma, M1770-1L) was prepared. This concentration was chosen to yield a total ion abundance for the standard that would be roughly equal to the abundance measured in the MTb samples. The headspace of the solution was extracted using SPME for five minutes, then run on the GC-MS dual-detector system with trap-and-purge. The 2-pentanone standard files are 12_{—}7, 8, and 9. The resulting TICs for these standards are shown in FIG. 13, and the mean retention time for 2-pentanone was 5.786±0.003 (% rsd=0.052%). Comparison of this result with the average retention time for Peak 4 (5.78₅±0.01₂) shows that there is no statistically significant difference between the retention times of the 2-pentanone standard and Peak 4 of the MTb samples at the 95% confidence level. This result supports the identification of Peak 4 as 2-pentanone. It is extremely unlikely that both 2-pentanone and the amide would coelute, given that the boiling point of 2-pentanone is 101-105° C. (Aldrich catalog) and has a formula weight of 86 while the amide has weight of 171.

The standard 2-pentanone mass spectra were also compared to that of the NIST library. In the case of 2-pentanone, there are at least three replicate entries for 2-pentanone in addition to the main library entry, and they are different enough to yield different scores when compared to known 2-pentanone. Variability in the known spectra thus is not considered cause for concern. For the three replicates of standard 2-pentanone, the spectral matches to the library for both 2-pentanone and for 2-methyl-N,N-diisopropylpropanamide are shown in Table O. Both forward and reverse searches against the NIST library for 2-pentanone reveal good matches between the spectra, however the reverse searches consistently earn a higher score than the forward searches. This result is expected, since each standard mass spectrum contains spurious peaks. The same trend is observed when the standard 2-pentanone is compared to the amide mass spectrum in the library, where the search scores are marginal to poor.

TABLE O
Library Matches for 2-Pentanone Standards

| File | Forward Search | Reverse Search | Probability Match |
|---|---|---|---|
| 12_7^{a} | 851 | 869 | 66.2 |
| 12_8^{a} | 817 | 841 | 61.9 |
| 12_9^{a} | 832 | 854 | 70.7 |
| 12_7^{b} | 658 | 671 | 0.06 |
| 12_8^{b} | 574 | 691 | 0.43 |
| 12_9^{b} | 647 | 668 | 0.04 |

^{a}= 2-pentanone
^{b}= amide

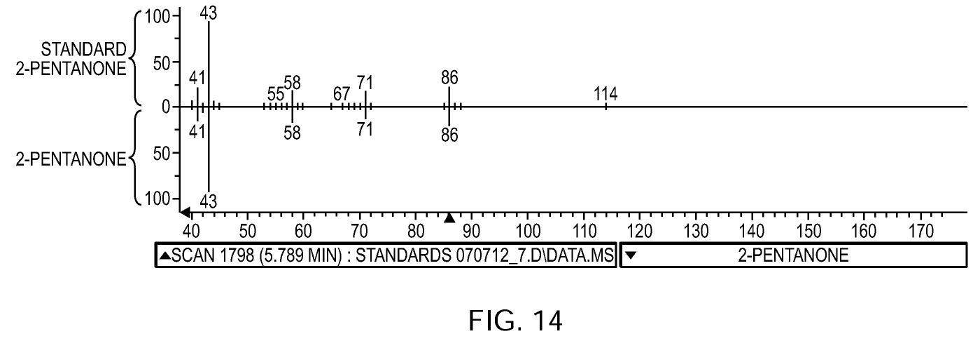

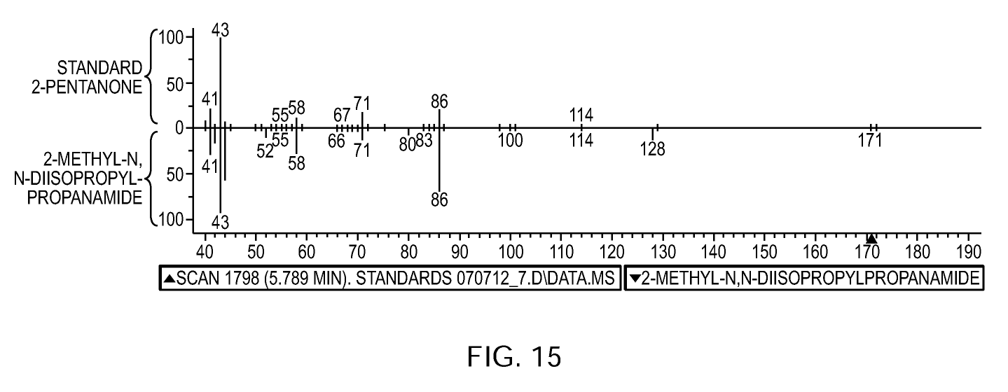

However, even when 2-pentanone is known to be present, the NIST library probability match does not consistently identify the compound with the desired level of confidence (FIG. 14). Comparison of the standard 2-pentanone mass spectrum with 2-methyl-N,N-diisopropylpropanamide (FIG. 15) shows poor correlation between these two compounds, which is reflected by poor probability matches. It is clear from FIG. 15 that the mass spectrum of 2-pentanone is a subset of 2-methyl-N,N-diisopropylpropanamide, with peaks observed at m/z values of 41, 42, 43, 44, 58, 71, 86 and 87. This may explain the reason that MTb Peak 4 frequently was identified as the amide instead of the ketone. Since the mass spectrum of 2-pentanone may be subsumed in the mass spectrum of the amide, it is reasonable to expect that its “uniqueness score” will not be high. Moreover, it is expected that 2-pentanone would score poorly in a forward search but more favorably in a reverse search. Further complicating the mass spectral identification is the presence of argon and carbon dioxide peaks in six of the eight MTb replicates.

Finally, evaluation of the mass spectra for the various MTb and 2-pentanone standard samples reveals that additional spurious peaks, which may come from any number of sources, do appear regularly. Direct injection of 2-pentanone via syringe yielded better than a 90% probability match. These spurious peaks are not common (i.e., reproducible) or consistent (i.e., the same m/z ratio) enough to suggest that more than one analyte is eluting at the same time. However, given the low abundance of ions in this peak, any anomalous peak may have deleterious effects on the spectral match. Evaluation of the spectra in FIG. 15 shows a minor peak is observed in the 2-pentanone standard at m/z=114. This peak is found in 2-methyl-N,N-diisopropylpropanamide at a comparable relative abundance, and as such reduces the quality of the match.

If the MTb Peak 4 were 2-pentanone, there should be no m/z peaks beyond 86, since this is the formula weight of the molecule. If peak 4 were the amide, according to FIG. 15, characteristic peaks would be expected at m/z values of 100, 114, 128, and 171, the parent ion of the amide. Based on the relative abundances of these peaks, at least two of these peaks should be consistently observed. However, evaluation of all eight MTb replicates showed no peak at 100, 114 or 128. Only MTb file 13_{—}9 showed a minor peak (total ion abundance of 10) at m/z=171 and only 2-pentanone standard file 12_{—}7 showed a minor peak at 114. Thus, the absence of characteristic, higher-weight ions from the mass spectra indicates that Peak 4 is not 2-methyl-N,N-diisopropylpropanamide.

TABLE P
Relative Ion Abundances for 2-Pentanone Mass Spectra in the Standards
and MTb Files

| | | m/z | | | | | | | |
|---|---|---|---|---|---|---|---|---|---|
| | | 43 | 86 | 71 | 41 | 58 | 42 | 44 | 40 |
| Stan- | 12_7 | 100 | 19.4 | 14.7 | 21.7 | 10.8 | 5.5 | 7.1 | 8.3 |
| dard | | | | | | | | | |
| Stan- | 12_8 | 100 | 24.2 | 14.8 | 10.9 | 7.6 | 6.2 | 9.2 | 7.6 |
| dard | | | | | | | | | |
| Stan- | 12_9 | 100 | 21.7 | 10.2 | 14.6 | 4.8 | 4.5 | 11.1 | 8.0 |
| dard | | | | | | | | | |
| MTb | 13_4 | 100 | 24.9 | 11.2 | 16.5 | 8.1 | 7.2 | 48.9 | 157 |
| MTb | 13_6 | 100 | 21.6 | 14.7 | 10.5 | 11.5 | 5.2 | 90.4 | 340 |
| MTb | 13_9 | 100 | 19.8 | 7.4 | 11.2 | 3.4 | 9.9 | 39.6 | 148 |
| MTb | 13_10 | 100 | 31.7 | 13.6 | 13.5 | 7.8 | 10.9 | 61.2 | 155 |
| MTb | 14_4 | 100 | 21.6 | 9.2 | 8.7 | 3.0 | 6.7 | 41.8 | 155 |
| MTb | 14_6 | 100 | 11.4 | 8.1 | 11.5 | 9.6 | 5.7 | 44.6 | 146 |
| MTb | 15_5 | 100 | 16.7 | 12.7 | 8.6 | 7.2 | 5.8 | 31.7 | 10.7 |
| MTb | 15_7 | 100 | 23.4 | 7.2 | 7.2 | 5.9 | 5.0 | 28.5 | 5.2 |

Next, the MTb mass spectra were compared to those of the standards, and the results are summarized in Table P. The m/z values for 2-pentanone are arranged in descending order. It is clear that the relative intensities of the m/z values for the amide do not descend in the same sequence, which allows one mechanism for differentiating between these two compounds in the MTb sample peak, and further indicates that Peak 4 is 2-pentanone.

2f. Detection of Methoxybenzene (Anisole) as a Compound Indicative of Mtb in a Sample.

Peak 5 presented several challenges in compound identification, partly due to the small signal (≦4000 total ion abundance) in the TIC. Five library reference compounds matched to various degrees with the TB peak: methoxybenzene (AKA anisole; MW=108), 7-deoxy-1-glycero-d-manno-heptonic phenylhydrazide (MW=300), 1-amino-2-methylpyridinium hydroxide (MW=108), α,d-gala-octonic phenylhydrazide (MW=346), and methyl phenyl carbonate (MW=152). Preliminary evaluation of the mass spectra suggested that the compound was methoxybenzene, and the NIST library results are summarized in Table Q.

TABLE Q
NIST Library Matches for MTb Peak 5 (tᵣ = 8.87₀ ± 0.01₃)
with Methoxybenzene

| File | Forward | Reverse | Probability |
|---|---|---|---|
| 13_4 | 576 | 670 | 15.7 |
| 13_6 | 454 | 550 | 1.24 |
| 13_9 | 562 | 663 | 9.66 |
| 13_10 | 583 | 679 | 5.08 |
| 14_4 | 548 | 691 | 8.68 |
| 14_6 | 553 | 669 | 40.3 |
| 15_5 | 667 | 788 | 68.3 |
| 15_7 | 688 | 819 | 72.4 |

Almost all forward, reverse, and probability matches for the TB mass spectra were poor, with the exception of files 15_{—}5 and 7. These results are not surprising based on the low peak intensity, the presence of spurious peaks, and the fairly large number of other library entries that match the mass spectrum.

Unique mass spectral features that differentiate methoxybenzene from the other library compounds were identified. On this basis and considering their large formula weights, α,d-gala-octonic phenylhydrazide and 7-deoxy-1-glycero-d-manno-heptonic phenylhydrazide were eliminated as possible matches. Most notably, the relative peak abundances for m/z=77 and 78 should be roughly 28% and 15%, respectively, for both of the phenylhydrazide compounds. This was not observed in the MTb mass spectra. Similarly, the relative abundance values for m/z=92 and 93 are roughly 38%-40% and 14-23%, respectively, for the phenylhydrazides. These ratios were not observed in the MTb mass spectra.

The NIST library entry for methyl phenyl carbonate indicates that the peaks at m/z=65 and 108 should have relative abundances of 100% and 42%, respectively. In the MTb peaks, these abundances are instead 64% and 100% (essentially the opposite). Additionally, the parent ion peak of methyl phenyl carbonate occurs at m/z=152 with a relative abundance of about 37%. This would be plainly visible in the MTb mass spectra, however no peak is observed for five of the eight replicates at this ratio. The three files that do show a peak at 152 have absolute ion abundance values between 13-17. Using the absolute ion abundance values for m/z=108 and an estimated relative abundance of 37% for m/z=152, this peak should have an absolute abundance between 90-200 in these mass spectra. The abundance values in the MTb files are too low to support a match to methyl phenyl carbonate. The final characteristic of methyl phenyl carbonate is the presence of a peak at m/z=59 with a relative abundance of 21%. This peak is completely absent in six of the eight replicates. Those spectra that do have this peak have relative abundance values of 5.3 and 9.0%, far from the expected value. These data do not provide compelling support for the peak identity as methyl phenyl carbonate.

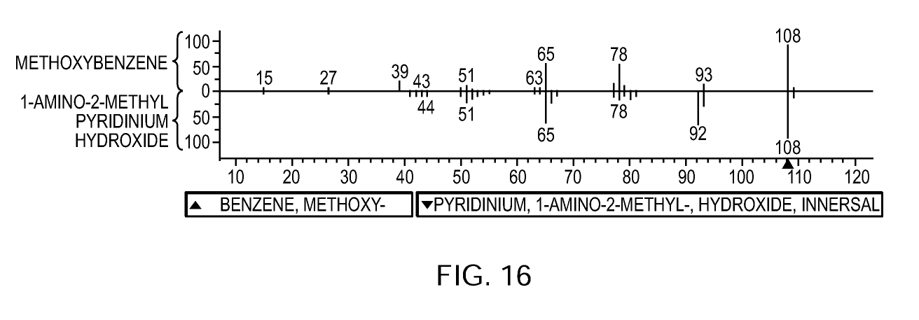

It is more challenging to distinguish between the mass spectra of methoxybenzene and the pyridinium salt, particularly in light of the fact that both compounds are aromatic and have the same molecular weight. As such, both compounds produce most of the same ion fragments in comparable relative abundances, as may be seen in FIG. 16.

The chief, usable differences between the two mass spectra in FIG. 16 are the presence of peak at m/z=39 for methoxybenzene and the relative abundances of peaks at m/z=92 and 93. For the pyridinium salt, the relative abundance values for these peaks are 60% and 27% respectively; methoxybenzene values are m/z=93 at 14% and m/z=92 at ˜5%. Evaluation of the MTb mass spectra show that six of the eight replicates have peaks at m/z=39 with an average of 28% (range: 13-40%) relative abundance versus about 20% in the reference spectra. Two of the spectra do not show this peak, as the m/z=39 is the lowest ion ratio measured by the instrument and doesn't always appear in every mass spectrum (i.e., often the x-axis begins at m/z=40, not 39). Next, if the MTb peak is the salt, the m/z=92 should be about twice as abundant as the m/z=93 peak. The average relative abundance of m/z=93 for the MTb replicates is 14% (range=7.5-20%), so there should be no difficulty measuring the peak at 92 if the peak is the pyridinium salt. This is not observed in any of the MTb mass spectra, where five of the eight replicates have no peak whatsoever at m/z=92; those three that do have a peak range in relative abundance from 2.8%-5.6%. These data indicate that the MTb Peak 5 is not 1-amino-2-methylpyridinium hydroxide.

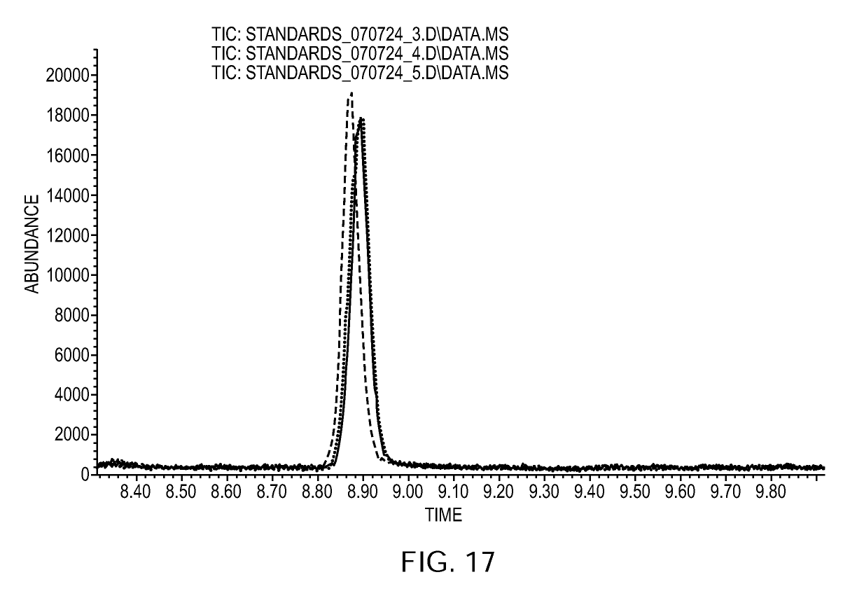

To validate the identity of Peak 5, methoxybenzene (anhydrous, 99.7%, Sigma-Aldrich) was prepared in methanol (LC-MS Chromasolv, Sigma-Aldrich) at a concentration of about 30 ppmv. This solution concentration yielded a total ion concentration of roughly 18,000, which is far greater than the 2000-4000 range seen in the MTb files. In the lower range, the methoxybenzene standards did not yield good matches to the NIST library, an observation consistent with the MTb files. The data files are 24_{—}3, 4, and 5, and the TICs for these files are shown in FIG. 17. The average retention time of methoxybenzene was 8.88₆±0.01₄ (% rsd=0.15%). This result compares most favorably to that of Peak 5, with a mean retention time of 8.87₀±0.01₃, and there is no statistically significant difference between the two retention times at the 95% confidence level. These data indicated that Peak 5 was methoxybenzene.

The standard spectra of methoxybenzene were next compared to the NIST library entries, and these results are summarized in Table R. The forward and reverse spectral matches for the standards were excellent, while the match probabilities were good.

TABLE R
NIST Library Matches for Methoxybenzene Standards

| File | Forward Search | Reverse Search | Probability Match |
|---|---|---|---|
| 24_3 | 920 | 924 | 88.1 |
| 24_4 | 901 | 914 | 84.7 |
| 24_5 | 894 | 912 | 82.2 |

The mass spectra of the standards were then compared to the MTb files (see Table S), and the relative abundance values between both data sets agree closely. The relative peak abundances show the following trend: 108>78≈65>39>77≈51≈79≈93, thus providing additional evidence in support of the identity of the peak.

TABLE S
Relative Ion Abundances for Methoxybenzene Mass Spectra in
the Standards and MTb Files

| | | m/z | | | | | | | |
|---|---|---|---|---|---|---|---|---|---|
| | | 108 | 78 | 65 | 39 | 77 | 51 | 79 | 93 |
| Stan- | 24_3 | 100 | 63.8 | 65.6 | 22.3 | 16.4 | 12.9 | 13.3 | 16.9 |
| dard | | | | | | | | | |
| Stan- | 24_4 | 100 | 69.5 | 69.4 | 22.0 | 19.4 | 14.5 | 14.0 | 16.1 |
| dard | | | | | | | | | |
| Stan- | 24_5 | 100 | 57.2 | 66.2 | 23.0 | 21.8 | 14.8 | 15.7 | 13.8 |
| dard | | | | | | | | | |
| MTb | 13_4 | 100 | 74.4 | 69.9 | 42.2 | 15.7 | 19.9 | 26.5 | 9.6 |
| MTb | 13_6 | 100 | 55.6 | 76.4 | 19.9 | 13.0 | 23.6 | 22.2 | 7.9 |
| MTb | 13_9 | 100 | 79.3 | 86.8 | NM | 35.8 | 15.5 | 43.8 | 17.9 |
| MTb | 13_10 | 100 | 45.4 | 46.9 | 32.7 | 11.9 | 14.2 | 18.2 | 17.3 |
| MTb | 14_4 | 100 | 59.2 | 91.1 | 39.5 | 38.8 | 11.8 | 22.4 | 20.4 |
| MTb | 14_6 | 100 | 52.2 | 42.3 | 13.4 | 11.7 | 22.4 | 11.1 | 7.5 |
| MTb | 15_5 | 100 | 56.0 | 42.9 | NM | 16.4 | 9.7 | 23.9 | 12.3 |
| MTb | 15_7 | 100 | 68.4 | 53.8 | 17.7 | 17.7 | 11.5 | 11.8 | 17.0 |

In Table S, it is worth noting that for two MTb files, no peak was measured at m/z = 39. This is not uncommon, as this value is the lowest m/z measured by the method and often does not appear in mass spectra. Based on these data, Peak 5 was identified as methoxybenzene (anisole).

2g. Detection of 2-ethylhexanoic Acid Methyl Ester (methyl 2-ethylhexanoate) as a Compound Indicative of MTb in a Sample.

MTb Peak 6 spectral matches for methyl 2-ethylhexanoate (MW=158) to the NIST library are shown in Table T. All forward searches and most of the reverse searches earned poor scores, while about half of the mass spectra earned fair probability match scores. The library contains three total entries (the main entry plus two replicates) for this compound. It is particularly worth noting that this peak typically has one of the lowest total ion abundances (≦4500) for each of the eight replicates, so that the total signal intensity in the mass spectra is less than desirable.

TABLE T
NIST Library Matches for MTb Peak 6 (tᵣ = 12.53₆ ± 0.02₁)
with Methyl 2-Ethylhexanoate

| File | Forward | Reverse | Probability |
|---|---|---|---|
| 13_4 | 615 | 689 | 64.8 |
| 13_6 | 419 | 455 | 0.65 |
| 13_9 | 504 | 565 | 49.4 |
| 13_10 | 583 | 627 | 51.5 |
| 14_4 | 614 | 693 | 72.2 |
| 14_6 | 547 | 648 | 63.1 |
| 15_5 | 650 | 795 | 72.7 |
| 15_7 | 616 | 801 | 71.9 |

Evaluation of the library mass spectra show the following sequence of relative abundance values for the principle m/z values: 102≈87>57>41>55>43≈101≈130. The MTb peaks agree reasonably well with these values, as shown in Table U, which supports the identification of Peak 6 as methyl 2-ethylhexanoate. Moreover, no other compound consistently and/or closely matched these mass spectra.

TABLE U
Relative Ion Abundances for Methyl 2-Ethylhexanoate Mass
Spectra in the MTb Files

| | | m/z | | | | | | | |
|---|---|---|---|---|---|---|---|---|---|
| | | 102 | 87 | 57 | 41 | 55 | 43 | 101 | 130 |
| MTb | 13_4 | 100 | 85.0 | 50.8 | 27.5 | 31.1 | 26.9 | 15.8 | 9.4 |
| MTb | 13_6 | 90.6 | 100 | 72.0 | 54.2 | 41.1 | 50.5 | 25.2 | NM |
| MTb | 13_9 | 62.0 | 100 | 44.4 | 44.0 | 18.8 | 33.8 | 9.8 | 13.7 |
| MTb | 13_10 | 100 | 71.4 | 74.3 | 52.9 | 31.6 | 57.3 | 18.4 | 6.3 |
| MTb | 14_4 | 100 | 94.4 | 64.5 | 44.5 | 29.0 | 29.4 | 18.2 | 31.6 |
| MTb | 14_6 | 97.1 | 100 | 71.0 | 46.5 | 28.1 | 35.0 | 26.7 | 21.2 |
| MTb | 15_5 | 100 | 91.8 | 49.6 | 13.1 | 21.7 | 20.1 | 18.0 | 10.7 |
| MTb | 15_7 | 100 | 100 | 56.7 | 33.5 | 44.2 | 29.9 | 20.5 | 8.9 |

During the course of these studies it was determined that methyl 2-ethylhexanoate has a chiral center. Certain organisms, such as MTb, may produce a single chiral version of this compound rather than a racemic mixture of this compound. Accordingly, depending on various factors, such as the source of the organisms, the variety of organisms in the sample, the metabolic states of those organisms, and, optionally, culturing conditions, the presence or absence of a single chiral version of this compound may be indicative of the presence of absence of MTb in a sample.

2h. Detection of Additional Aromatic Compounds

Samples were collected and analysis conducted as generally described above to identify additional volatiles indicative of the presence of MTb in a sample, including methyl 2-methylpropionate (CAS: 547-63-7), 2,4-dimethyl-1-heptene (CAS: 19549-87-2), methyl isobutyl ketone (CAS: 108-10-1), 6-methyl-5-hepten-2-one (CAS: 110-93-0), dimethylsulfoxide (CAS: 67-68-5), dimethylsulfide (CAS: 75-18-3), 1-ethoxy-2-methylpropane (CAS: 627-02-1), 1-ethoxy-butane (CAS: 628-81-9), t-butyl ethyl ether (CAS: 637-92-3), isobutanol (CAS: 78-83-1), and the aromatic compound represented by the mass spectrum in FIG. 22. The specific analytical conditions used to obtain mass spectrum in FIG. 22 are described in detail in Example 4 below.

### Example 3

This example describes a method for identifying particular bacteria (e.g., S. aureus, K. pneumonia, and E. coli) in a sample using data from both GC-MS analysis and DMS analysis. Accordingly, this example also describes a library of data and a method for generating a library of data for a point-of-care diagnostic tool to facilitate rapid identification of the bacteria in a sample. A micromachined DMS device is an example of a point-of-care diagnostic tool. The DMS device is portable and the detection methodology is capable of low limits of detection of analytes.

Specifically, simultaneous analysis by GC-MS and DMS was used to identify medically important bacteria in a sample, including Staphylococcus aureus, Klebsiella pneumonia and Escherichia coli. A dual system platform allowed for the correlation of the spectral pattern of the bacterial VOCs in DMS with the identification of each compound structure with GC-MS, and generation of a DMS data library to allow for identification of the bacteria based on DMS data alone. This process also led to the discovery of several VOCs that are biomarkers for each particular bacteria. Utilization of the identified biomarkers for S. aureus enabled the identification of its presence in a mixed culture with other medically important bacteria.

This system represents a powerful platform wherein discovery of novel VOCs may be applied to detection with DMS. The ability to rapidly identify and speciate medically important bacterial pathogens is one application of this platform and can enhance medical care for life threatening infections.

3a. Background

Current clinical laboratory methods to identify medically important bacteria depend on well established microbiological techniques that are relatively slow, usually requiring several days for speciation. The unique biochemical pathways of different types of bacteria have been well studied and exploited for bacterial speciation tests. These form the basis for clinical identification platforms such as the API test strips (bioMerieux Vitek, Inc; Marcy-lEtoile, France) which are commonly used to speciate bacterial isolates. More recent technologies for speciating bacteria largely depend on nucleic acid based detection methods such as polymerase chain reaction (PCR), which are expensive, require significant expertise to perform, are often limited to the types of organisms detectable within a multiplex format, and are at risk for producing false positive results as a result of nucleic acid contamination. Therefore, there is a need for new techniques which combine the rapidity and sensitivity of PCR with the simplicity, specificity and broad applicability of traditional culture based techniques.

The diverse set of metabolic pathways utilized by different bacterial species result in production of a unique set of compounds that are byproducts of their metabolism. A subset of these metabolic byproducts, VOCs, are strong candidate biomarkers for diagnostic detection. VOCs are compounds that have a low vapor pressure and a low water solubility. VOCs may be detected by a variety of techniques, including many readily being sensed by human olfactory pathways, and represent the basis for medical technologists being able to identify bacteria by their smell. However, GC-MS remains the gold standard for detecting VOCs because of (1) sensitivity and (2) spectrometric information about the mass or mass fragments may be used to determine molecular structure. Unfortunately, current GC-MS technology is not viable for a point-of-care application in the field or even outside of controlled environments such as advanced clinical laboratories. This is due to, among other things, the relative lack of portability, significant power consumption and advanced maintenance requirements.

The DMS device is a micromachined sensor that has been shown to detect selected VOCs down to parts per trillion. However, in contrast to GC-MS, it is portable and relatively inexpensive. Previous studies with DMS have shown the ability to recognize compounds, distinguish classes of bacteria using pattern recognition, and detect chemical or biowarfare agents with high reliability. However, there is a significant concern of false positive detection given that VOCs may be detected in trace amounts. Furthermore, pattern recognition algorithms to recognize shared DMS signatures have been difficult to apply to clinical diagnostics given the lack of adequate specificity when comparing DMS signatures from different bacteria and from background analytes.

In various embodiments, the present invention successfully uses DMS to identify particular bacteria in a sample by first developing and employing a dual detection system that identifies compounds with DMS simultaneously with GC-MS. Thus, VOCs that are biomarkers for bacteria or other processes may be rapidly identified by GC-MS and the corresponding spectral pattern of DMS may be determined simultaneously and stored in a library for later reference. Once these identifications are collected in a DMS data library, the DMS spectral patterns may then be used for bacterial speciation by DMS alone. The following procedures describe the development of the dual detection system for biomarker determination and show how this platform may be used to detect the presence, amount, and/or state (e.g. viable, growing, etc.) of a particular bacteria and/or related bacterial strain in a complex clinical sample containing several different species of bacteria, using only data from a point-of-care diagnostic tool. In addition, the following procedures were used to identify and characterize novel biomarkers for particular bacteria.

3b. Methods and Materials
3b1. Instrumentation

Chromatographic separation of VOCs was achieved using an Agilent 6890N (Agilent Technologies; Palo Alto, Calif.) gas chromatograph equipped with an Rtx®-200MS trifluoropropylmethyl polysiloxane column (30 m×0.32 mm I.D., 1 μm film thickness; Restek Corporation; Bellefonte, Pa.). Chromatographic performance was monitored for consistency several times each day by use of a standard solution. The GC injection port, lined with a SPME injection sleeve (0.75 mm I.D.; Supelco, Bellefonte, Pa.), was operated at a constant temperature of 250° C. in splitless mode with a purge delay of 2 minutes. The front of the GC column was cooled cryogenically (Cryogenic Enrichment System CTE; GERSTEL; Baltimore, Md.) with liquid nitrogen to −125° C. for 2 minutes while the GC oven was held at 50° C. The cryotrap was subsequently ramped at 20° C./second to 240° C. while the oven was initially ramped at 10° C./minute to 67° C. Following a 6 minute hold, the oven temperature was raised at 10° C./minute to 100° C., then 20° C./minute to a final temperature of 230° C., which was held for 3 minutes.

A Press-Tight® Y-connector (Restek Corporation; Bellefonte, Pa.) was used to direct the column eluate to both an Agilent 5975 quadrupole mass spectrometer (Agilent Technologies; Palo Alto, Calif.) and a differential mobility spectrometer (Model SVAC-V, Sionex Corporation; Bedford, Mass.) via guard columns of 0.25 mm I.D. and 0.5 mm I.D. The different guard columns were selected to ensure even splitting of eluate despite a disparity in the operating pressures of the two detectors. The transfer line to the DMS sensor was heated to 180° C. to prevent condensation along the segment between the GC oven and the DMS sensor. The mass spectrometer was operated in electron impact ionization mode scanning m/z values of 39-300 at a rate of 5.25 cycles/second. A tune was carried out daily on the mass spectrometer using PFTBA (Aglient Technologies; Palo Alto, Calif.). DMS analysis was performed by scanning compensation voltages from −26 V to +8 V at 0.65 scans/second while the dispersion voltage was held at 1100 V and the sensor temperature was set at 85° C. Nitrogen was used as the drift gas at a flow rate of 400 mL/minute.

3b2. Preparation of Bacteria Cultures

ATCC strains of E. coli (ATCC#25924), Staphylococcus aureus (ATCC#25923 methicillin sensitive), and Klebsiella pneumonia (ATCC# 13883) were grown at 37° C. overnight on trypticase soy agar 5% sheep blood plates (Remel, Lenexa, Kans.). Colonies were suspended in Enteric Fermentation Base media ((EFM, Becton Dickinson, Sparks, Md.) supplemented with 1% glucose and diluted to obtain liquid cultures of a specified optical density (OD₆₀₀=0.1). These were incubated at 37° C. with shaking (200 rpm) until the bacterial growth reached mid-log phase (approximately 2 hours).

3b3. Extraction Procedure

Solid phase microextraction fiber/holder assemblies were pre-equilibrated to extraction temperature in a 40° C. oven. To prepare six replicate samples of each bacterium, the cultures were dispensed in 2 mL aliquots into 10 mL SPME headspace vials (Supelco Inc.; Bellefonte, Pa.) and sealed with screw caps fitted with septa. Control samples were prepared by pipetting 2 mL of pure 1% EFM media into each of six headspace vials. Mixed samples consisted of equal volumes of bacteria at equal optical density (OD₆₀₀=0.3). The vials were agitated on an orbital shaker (medium speed) for 15-20 minutes at room temperature, then removed and placed in a custom-made vial rack designed to support the fiber assemblies during extraction. The SPME fibers were uncapped and pierced through the septum of each vial. Once the rack was placed in a 40° C. oven, the fibers were exposed to the headspace of the cultures/controls for 1 hour. At the end of the extraction period, the fibers were retracted, removed from the vials, and capped. The fiber assemblies were stored at 2-8° C. until analysis by GC/MS and DMS.

Headspace extractions were carried out using 50/30 μm divinylbenzene/Carboxen/Polydimethylsiloxane (DVB/Carboxen/PDMS) SPME fibers (Supelco Inc.; Bellefonte, Pa.) mounted in TuffSyringe™ holders (Field Forensics; St. Petersburg, Fla.) to facilitate handling. The fiber needles were fitted with custom-made PTFE tips to limit contamination during transport and storage. As by recommendation of the manufacturer, the fibers were conditioned in a GC injection port at 250° C. to 270° C. for 1 hour prior to initial use and after each analysis to prevent carryover effects.

3c. Results
3c1. Set Up of Dual System

In order to facilitate rapid determination of bacterial-specific VOCs while determining the DMS specific spectral pattern, a dual detection system was implemented in accordance with the flowchart shown in FIG. 1B. To facilitate rapid identification of complex mixtures, a cryo-focussing setup was used at the proximal end of the GC columns. This enabled better resolution of the collected VOCs. A gas chromatography instrument was set up with a with a 30 meter Restek column that was chosen based on its ability to separate VOCs. The distal end of the column was split with a Y-connector in order to transfer the column eluant simultaneously to the DMS and MS. A narrower ID guard column was used to transfer the eluant to the MS in order to dampen the vacuum pull from the MS as the DMS operates at atmospheric pressures. Flow rates were measured to confirm that the split was evenly distributing the eluant to both sensor platforms. The transfer line to the DMS was heated to insure that no condensation occurred from the sample eluant. Simultaneous triggering of the MS and DMS was done during introduction of the sample to insure that the start times were synchronized between the collection parameters of the MS and DMS software.

The retention times for introduced analytes were approximately the same although minor variation occurred due to the differences in transfer line length. The variance in retention time between the two sensor outputs was always less than 5%, thus enabling the determination of the corresponding DMS peaks and MS peaks.

3c2. Optimization of Extraction Parameters

Once the parameters of the dual detection platform were established, the system was used in conjunction with SPME in order to identify bacteria-specific volatile organic compounds. As there are many types of SPME coatings, several types were tested in order to identify the optimal coating. The three phase fiber has been previously identified as being able to extract the broadest range of VOCs including polar, non-polar and semi-volatile compounds. Testing confirmed this by showing that this fiber had a larger number of different compounds that it extracted when compared to other SPME coatings. For example, the PDMS/DVB/Carboxen fiber had on average 350 unique volatiles compared to 200 for PDMS/DVB fiber. Further optimization of the extraction temperature and time was performed which showed that a 1 hour extraction at 37° C. yielded approximately the same number of peaks as higher temperatures and longer extraction times. Even though temperatures greater than 70° C. may enhance volatility of compounds, this was not done because it could destroy the bacteria and thereby yield VOCs that were not relative to robust metabolism.

3c3. Identification of Bacterial Biomarkers

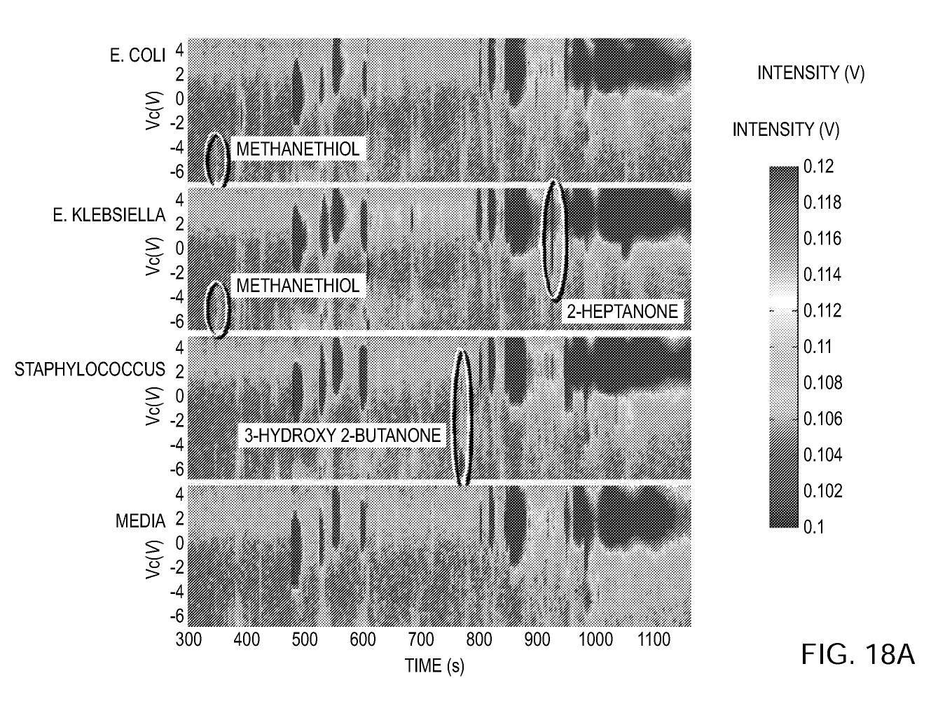

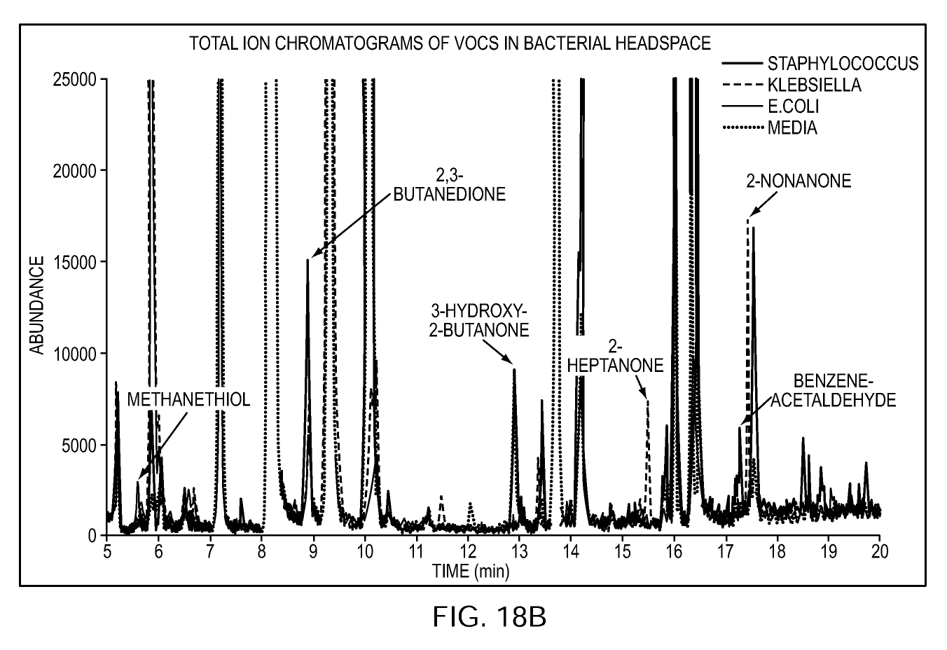

Once the sample preparation and instrumentation parameters were optimized, bacterial biomarker identification was performed with the dual GC-MS/DMS system. Medically important bacteria were grown on enteric fermentation media with a 1% glucose carbon source. Six replicates of each bacteria were prepared in order to control for sample to sample variation and obtain sufficient numbers of samples for accurate abundance calculations. Three different species of medically important bacteria—E. coli, S. aureus and K. pneumonia— were grown from colonies to an OD600 of 0.6 so that they were in log phase growth. All experiments were performed with media controls. In addition, blank fibers were also deployed in the working airspace in order to control for VOCs that might contaminate the fibers from an ambient source. The VOCs were then extracted and analyzed using the GC-MS and DMS instruments with the samples randomized into blocks to avoid confounding errors. The outputs were analyzed for unique compounds present in the bacterial cultures but absent in the media only or air controls. FIGS. 18A and 18B show exemplary DMS and chromatographic outputs from DMS and GC-MS, respectively, for three different bacteria and a control. In the figures, exemplary VOC peaks indicative of the respective bacteria are shown in circles (FIG. 18A) or by arrows (FIG. 18B). A comparison of the DMS data (FIG. 18A) and the GC-MS data (FIG. 18B) outputs shows that peaks indicative of particular bacteria in a sample can be more apparent in one analysis versus the other, depending on the noise or other signals that surround or overlap a signal from a candidate compound. Accordingly, in various embodiments, a VOC indicative of a particular bacteria (e.g., MTb, Staph, Kleb, or E. coli) in a sample may be detected through only one analytical technique, such as DMS or GC-MS, or through more than one analytical technique.

Once candidate compounds were determined from NIST, using matching statistics as well as forward and backward analysis (similar to methods described in the previous examples), candidate standards were ordered and their identity confirmed by running the standards at the same conditions. Table V lists exemplary volatile biomarkers that had medium or high confidence in identification, for three different types of bacteria.

TABLE V
Exemplary VOCs - Biomarkers for particular bacteria

| | | |
|---|---|---|
| compound | CAS | Confidence in ID |

Staphylococcus aureus

| | | |
|---|---|---|
| methanethiol | 74-93-1 | high |
| dimethyl sulfide | 75-18-3 | high |
| 2,3-butanedione | 431-03-8 | medium |
| 3-hydroxy-2-butanone | 513-86-0 | medium |
| butyl acetate | 123-86-4 | high |
| benzeneacetaldehyde | 122-78-1 | high |

E. Coli

| | | |
|---|---|---|
| methanethiol | 74-93-1 | high |
| dimethyl disulfide | 75-18-3 | high |
| indole | 120-72-9 | high |

Klebsiella

| | | |
|---|---|---|
| methanethiol | 74-93-1 | high |
| 2-heptanone | 110-43-0 | high |
| 2-nonanone | 821-55-6 | high |
| 2-undecanone | 112-12-9 | medium |

3c4. Complex Mixture Experiment

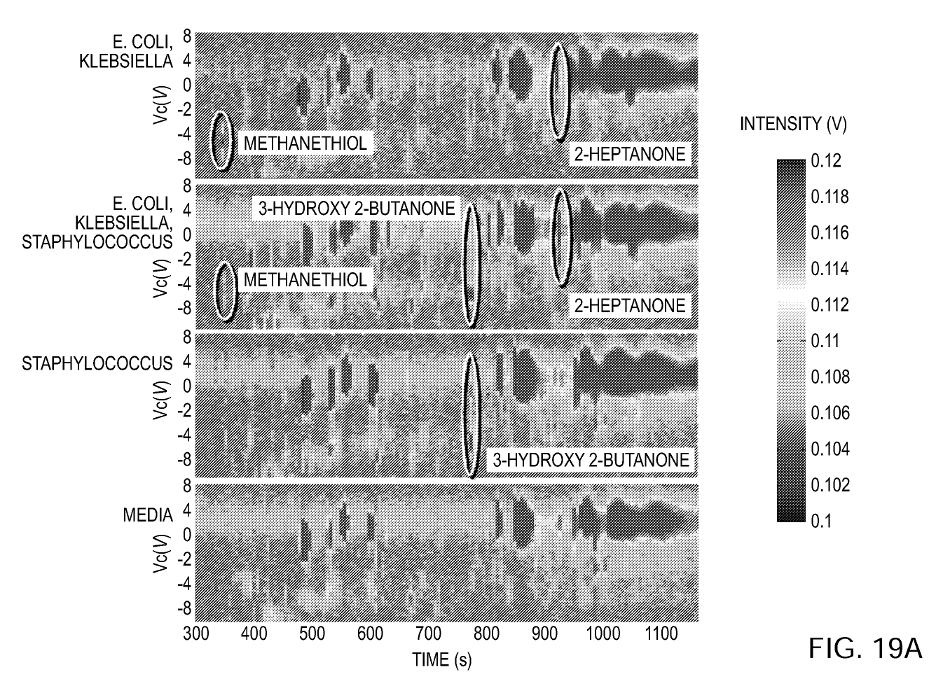

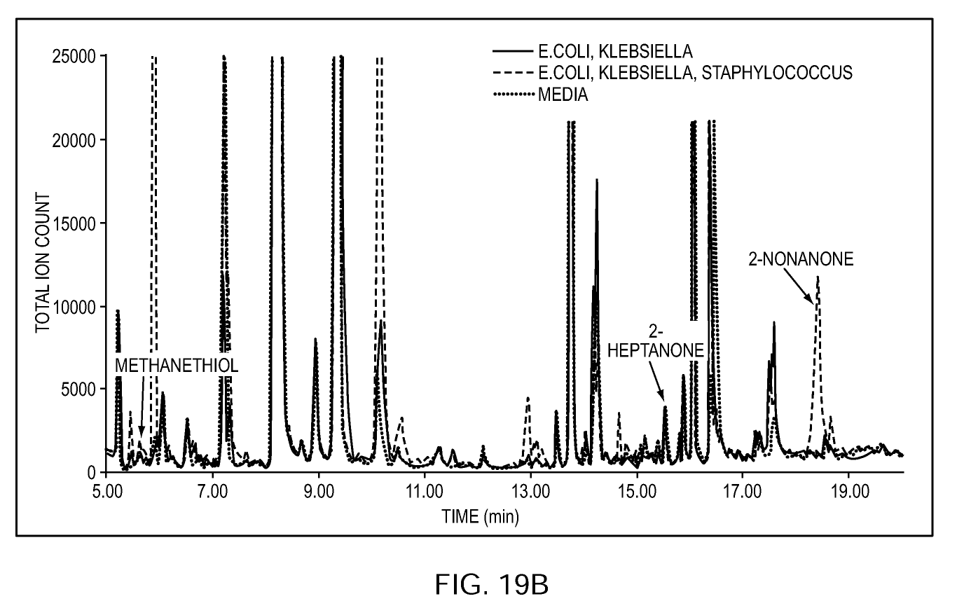

For detection of particular bacteria in a complex mixture, such as sputum or blood, it is important to show the ability to detect the particular bacteria from different bacteria that may contaminate the sample. For example, sputum samples are routinely contaminated with oral flora such as Streptococcus viridans, non-pathogenic Neisseria, and various anaerobic bacteria (see, e.g., Manual of Clinical Microbiology, 9^{th} Edition). In order to show that specific bacterial VOCs and/or combinations of VOCs are able to identify a bacteria in a mixture of bacteria, cultures were prepared containing equal mixtures of Klebsiella pneumonia and Escherichia coli with or without Staphylococcus aureus, an aggressive pathogen requiring rapid identification in a clinical setting to optimize patient outcome. FIGS. 19A and 19B show exemplary DMS and chromatographic outputs from DMS and GC-MS, respectively, for a mixture of E. coli and K. pneumonia, (ii) a mixture of S. aureus, E. coli and K. pneumonia, (iii) S. aureus alone, and (iv) media control. In the figures, exemplary VOC peaks are shown in circles (FIG. 19A) or by arrows (FIG. 19B). Analysis of these mixtures showed that S. aureus could be distinguished from E. coli and K. pneumonia, even by visual inspection of biomarker specific DMS data (see FIGS. 19A and 19B, and Table W). Subsequent analysis of the accuracy of several algorithms to distinguish the cultures containing S. aureus using DMS data showed a 100% accuracy suggesting that these biomarkers enables identification of S. aureus directly from clinical isolates with the portable DMS sensor.

TABLE W
Detection of bacteria in complex mixtures

| | | | |
|---|---|---|---|
| tᵣ | compound | CAS | Confidence in ID |

Staph

| | | | |
|---|---|---|---|
| 5.86 | methanethiol | 74-93-1 | high |
| 6.58 | dimethyl sulfide | 75-18-3 | high |
| 9.15 | 2,3-butanedione | 431-03-8 | medium |
| 12.75 | 3-hydroxy-2-butanone | 513-86-0 | medium |
| 12.88 | 3-methylbutanoic acid | 503-74-2 | low |
| 13.35 | butyl acetate | 123-86-4 | high |
| 22.52 | benzeneacetaldehyde | 122-78-1 | high |

E. Coli and Klebsiella

| | | | |
|---|---|---|---|
| 5.65 | sulfur dioxide | #7446-09-5 | high |
| 5.85 | methanethiol | 74-93-1 | high |
| 11.25 | 2-pentanone | 107-87-9 | medium |
| 13.35 | butyl acetate | 123-86-4 | high |
| 17.19 | 2-heptanone | 110-43-0 | high |
| 13.35 | 2-nonanone | 821-55-6 | high |

Staph, E. Coli, and Klebsiella

| | | | |
|---|---|---|---|
| 5.62 | sulfur dioxide | #7446-09-5 | high |
| 5.83 | methanethiol | 74-93-1 | high |
| 9.08 | 2,3-butanedione | 431-03-8 | medium |
| 11.17 | 2-pentanone | 107-87-9 | medium |
| 12.65 | 3-hydroxy-2-butanone | 513-86-0 | medium |
| 13.3 | butyl acetate | 123-86-4 | high |
| 17.14 | 2-heptanone | 110-43-0 | high |
| 23.32 | 2-nonanone | 821-55-6 | high |

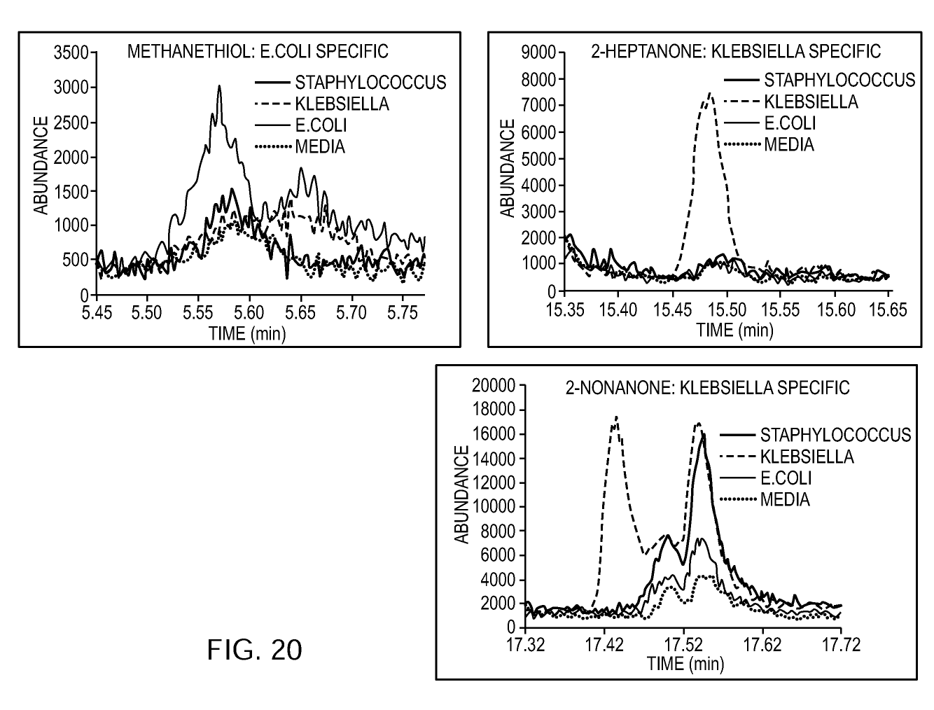

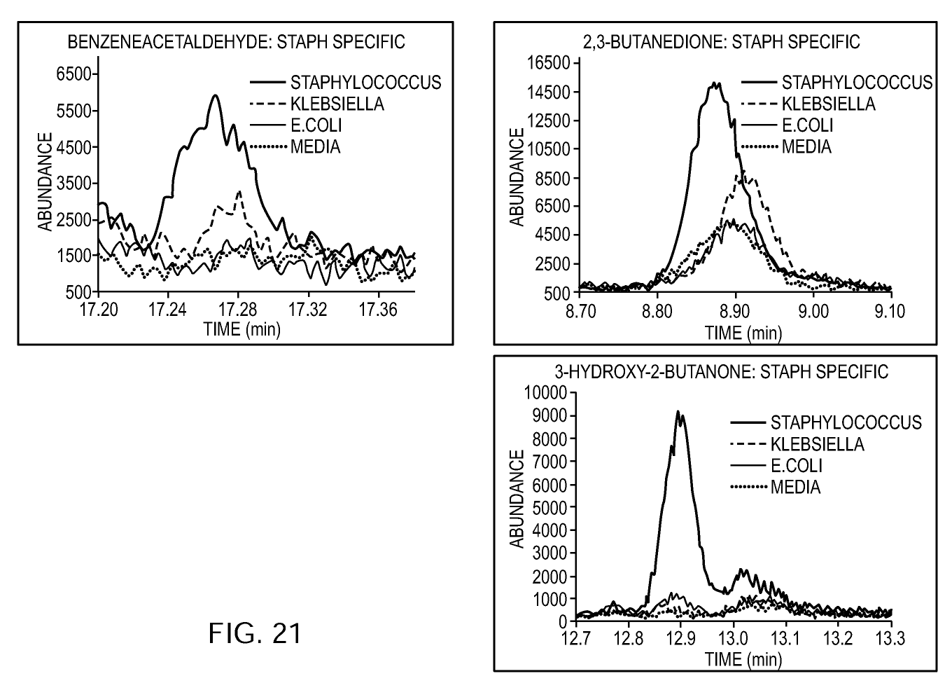

Specifically, the VOCs that are detected in a complex sample may be de-convoluted to identify particular bacteria within the sample. As shown in Table X, in a complex mixture of E. coli, S. aureus, and K. pneumonia, S. aureus may be identified by detecting 2,3-butanedione, 3-hydroxy-2-butanone, and/or benzeneacetaldehyde in the sample. K. pneumonia may be identified by detecting 2-heptanone and/or 2-nonanone in the sample. Similarly, the absence of a particular bacteria from a complex sample may be determined. For example, the absence of methanethiol from a sample indicates that E. coli and K. pneumonia are absent from the sample. Exemplary sections of GC-MS chromatograms that show E. coli specific, K. pneumonia specific, or S. aureus specific VOCs are shown in FIGS. 20 and 21. Specifically, depending on the other organisms in a sample, methanethiol can be specific for E. coli (or Kleb); 2-heptanone or 2-nonanone can be specific for K. pneumonia, and 3-hydroxy-2-butanone, benzeneacetaldehyde, or 2,3-butanedione can be specific for S. aureus. In addition, the absence of such compounds in a sample can be indicative of the absence of the corresponding bacteria in the sample.

TABLE X
Bacteria-specific VOCs in a complex sample

| | | Confidence in | | | | |
|---|---|---|---|---|---|---|
| Compound | CAS # | ID | E. Coli | Staphylcoccus | Klebsiella | Media |
| methanethiol | 74-93-1 | High | Y | ND | Y | ND |
| 2,3-butanedione | 75-18-3 | Medium | ND | Y | ND | ND |
| 3-hydroxy-2-butanone | 513-86-0 | Medium | ND | Y | ND | ND |
| benzeneacetaldehyde | 122-78-1 | High | ND | Y | ND | ND |
| 2-heptanone | 110-43-0 | High | ND | ND | Y | ND |
| 2-nonanone | 821-55-6 | High | ND | ND | Y | ND |

Y = detectable;
ND = not detectable.

3d. Discussion
3d1. Volatile Biomarker Approach

The experiments describe, in part, the setup and application of a dual detection system for identification of bacterial VOC biomarkers. VOCs are a powerful diagnostic biomarker because the detection of gas, for example headspace gas from a sample, requires much less sample processing time and expertise as compared to conventional methods that require isolation and identification of nucleic acids or proteins. However, because VOCs are often present in trace amounts (parts per billion and lower) there is a potential for contamination and or misidentification. Therefore, using pattern recognition algorithms alone without prior, simultaneous, and/or subsequent identification of the compound structure could introduce error, particularly for applications of the technology to complex samples. In order to address this problem, the compounds that were detected with the DMS sensor were simultaneously identified with GC-MS to confirm the identity of the compounds and generate a data library for a DMS point-of-care device that may be referenced to identify subsequent samples using the DMS point-of-care device alone. The power of a point-of-care device lies in its portability, accuracy, and speed. Using the dual detection platform to establish a data library for a point-of-care device allows for the point-of-care format to be used and give results directly in a clinical and/or field setting.

3d2. Applications Including In Vitro Diagnosis, Breath Analysis, and Environmental Monitoring

The data above show the ability of the DMS system to identify biomarkers for the medically important bacteria, Staphylococcus aureus. Furthermore, these biomarkers were utilized to identify with high accuracy the presence of Staphylococcus aureus in a mixture of several bacteria. This shows the ability to identify bacteria from mixed samples using a point-of-care device. Therefore, if the limits of detection are low, and the ability to distinguish the VOCs from background volatiles is sufficient, this technology may enable rapid species determination of bacteria from various sources, for example blood, sputum, soil, water, industrial products, and/or industrial waste streams. For example, embodiments of the present invention may be used for breath analysis of pulmonary infections. Staphylococcus aureus is known to be a significant pathogen involved in hospital-acquired or ventilator-associated pneumonia. If one were able to detect the presence or increase volatile biomarkers for this bacteria, then early intervention could save lives and considerable health care costs associated with these infections and contaminations.

Further characterization of volatile biomarkers from bacteria in simulated clinical specimens and validation of a library of bacterial markers may be performed by the methods described herein. Because of the portability of the DMS device, the application of this detection platform in the field is feasible without the need for a central microbiology laboratory. For example, a small clinic or even an elder care setting may diagnose a bacterial specific pneumonia using the methods described herein. Furthermore, application of DMS biomarker analysis to specimens or breath samples also enables the platform to detect host disease processes simultaneously, as the point-of-care data library may be developed and used by clinicians to detect a broad classes of host and pathogen-specific VOCs.

### Example 4

This example shows that certain VOCs consistently can be associated with a particular bacteria across different types of culture media. In addition, this example shows that in certain cases bacterial VOC expression can depend on the composition of the culture medium. These approaches can be useful for an improved method for the detection and/or quantitation of bacteria in a sample and for identification of VOCs that are culture-media specific and VOCs that are not culture-media specific.

### 4.1 Culturing

Mycobacterium tuberculosis strain H37Rv was maintained on Middlebrook 7H10 agar or 7H9 broth supplemented with 10% OADC enrichment. The cells were pelleted at 4000 rpm for 5 minutes, washed with phosphate buffer solution and resuspended in minimal media (0.5 g/L asparagine, 1 g/L KH₂PO₄, 2.5 g/L Na₂HPO₄, 50 mg/L ferric ammonium citrate, 0.5 g/L MgSO₄*7H₂O, 0.5 mg/L CaCl₂, 0.1 mg/L ZnSO₄) containing palmitate (0.1% w/v), glycerol (0.1%), cholesterol (0.01%) or sodium propionate (0.1% to 10%). The resulting cultures were dispensed into headspace vials in 1 mL aliquots to create five or six replicates of each. Uninoculated control cultures were generated by pipetting pure media into vials. All samples were grown at 37° C. degrees overnight and then subjected to SPME headspace extraction for 30 minutes at 37° C.

### 4.2 Sample Analysis

Chromatographic separations were achieved using an Agilent 6890N (Agilent Technologies; Palo Alto, Calif.) gas chromatograph equipped with a Rtx®-200MS trifluoropropylmethyl polysiloxane column (30 m×0.32 mm I.D., 1 μm film thickness; Restek Corp.; Bellefonte, Pa.). The GC injection port was fitted with a Merlin Microseal (Supelco Inc.; Bellefonte, Pa.) and lined with a 0.75 mm I.D. SPME injection sleeve (Supelco Inc.). The inlet was set at a temperature of 270° C. (DVB/Carboxen/PDMS fibers) or 300° C. (Carboxen/PDMS fibers) with a constant operating pressure of 4.75 psig; it was run in splitless mode during analyte desorption (2 minutes) followed by split mode (20 mL/minute split flow) for the remainder of the GC program. A column flow rate of 2.0 mL/min was maintained throughout. Bottled helium (99.99% purity) scrubbed over a SGT triple filter (Restek Corp.) was used as the carrier gas. The proximal end of the GC column was cooled cryogenically (Cryogenic Enrichment System CTE; GERSTEL; Baltimore, Md.) with liquid nitrogen to −125° C. during desorption, then ramped at 20° C./s to 240° C. for a 2 minute hold. The main oven temperature program was as follows: initial temperature of 50° C., hold for 2 minutes, ramp to 67° C. at 10° C./min, hold for 6 minutes, ramp to 10° C./minutes to 100° C., then ramp to 230° C. at 20° C./minutes, and hold 3 minutes.

A Press-Tight® Y-connector (Restek Corp.) was used to simultaneously direct the GC column eluate to both an Agilent 5975 quadrupole mass spectrometer (Agilent Technologies; Palo Alto, Calif.) and a differential mobility spectrometer (Model SVAC-V, Sionex Corporation; Bedford, Mass.) via guard columns of 0.25 mm I.D. and 0.5 mm I.D. respectively. These guard columns were selected to compensate for the disparity in detector operating pressures—DMS units run at atmospheric pressure while the MS detector operated at approximately 5×10⁻⁷ torr. Empirical flow rate measurements confirmed that the column eluate was evenly disbursed to the two sensor platforms. To prevent condensation, the transfer line to the DMS was heated to 180° C. using flexible heating tape and a variable transformer. Nitrogen served as the DMS drift gas with a flow rate of 400 mL/minute. DMS data collection proceeded while scanning compensation voltages from −26 V to +8 V at a rate of 1.28 sec/scan. The DMS dispersion voltage was held at 1100 V, and the ⁶³Ni source was operated at 85° C. The MS spectra were recorded in full scan mode over a range of 39-300 m/z at a rate of 5.25 cycles/s. MS and DMS data collection was simultaneously triggered at the start of analyte desorption to ensure synchronization with respect to the time domain.

### 4.3 Bacterial VOC Expression in Different Culture Media

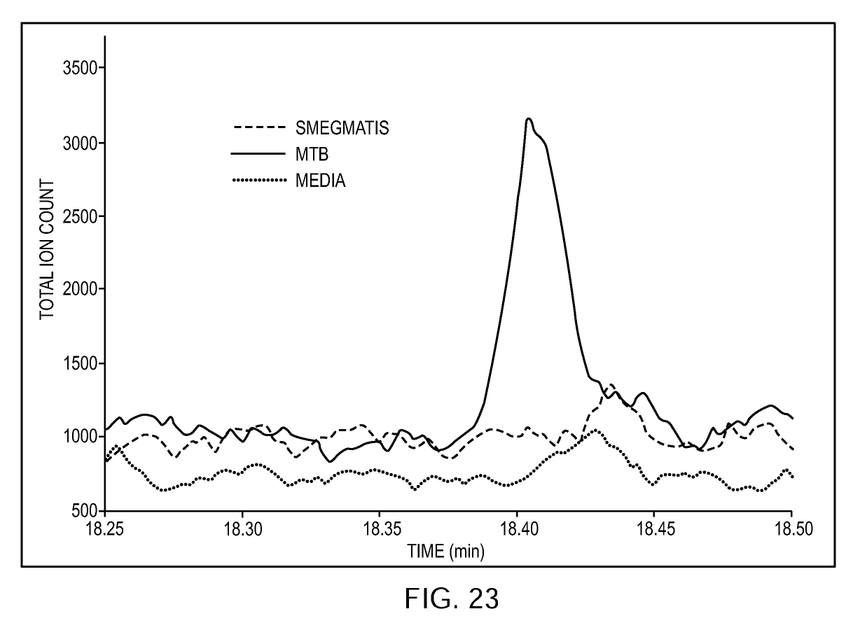

In one set of experiments, sample culture media each contained one different lipid-type carbon source, namely: cholesterol, palmitate, or sodium propionate. MTb was cultured on each medium, and the cultures were shaken to enhance the availability of oxygen to the cells. The extracted VOCs were detected using methods described above. Resulting data is shown in Table Y. Methyl-2-ethyl hexanoate and an additional aromatic compound having the MS spectra shown in FIG. 22 were detected in each of the three media types. As shown in FIG. 23, the aromatic compound having the MS spectra shown in FIG. 22 was not observed in the media, nor in smegmatis cultures prepared under the same conditions. Specifically, FIG. 23 shows the peak of the aromatic compound having the MS spectra shown in FIG. 22 having a retention time at approximately 18.44 minutes using the analytical method described above. This peak is present in the Mtb sample but not in the sample control or a sample of smegmatis.

These experiments show that certain VOCs, such as methyl-2-ethyl hexanoate and the additional aromatic compound having the MS spectra shown in FIG. 22, are associated with a particular bacteria across different culture media. Alternatively, certain VOCs, such as methyl propionate and 3-pentanone, are associated with a bacteria in a single culture media (i.e., are present when propionate is present in the media).

TABLE Y
MTb cultures, shaken, on three different media lipids

| | | | | | Retention | Retention | |
|---|---|---|---|---|---|---|---|
| | | | | | Time | Time of | Confidence |
| Compound | CAS # | Cholesterol | Palmitate | Propionate | (min) | Standard | in ID |
| methyl | 554-12-1 | − | − | + | 8.25 ± 0.04 | 8.24 ± 0.04 | high |
| propionate | | | | | | | |
| 3-pentanone | 96-22-0 | − | − | + | 11.21 ± 0.05 | 11.21 ± 0.04 | high |
| methyl-2- | 816-19-3 | + | + | + | 16.23 ± 0.01 | 16.22 ± 0.01 | high |
| ethyl | | | | | | | |
| hexanoate | | | | | | | |
| Additional | | + | + | + | 18.44 ± 0.01 | ND* | ND* |
| aromatic | | | | | | | |
| compound | | | | | | | |

*= Not Determined

Overall, the VOC data from these experiments show that particular VOCs, namely methyl propionate, 3-pentanone, methyl-2-ethyl hexanoate, and the additional aromatic with the spectral pattern shown in FIG. 22, consistently were detected in MTb cultures having a propionate-based media. The particular media sources of cholesterol, palmitate, and propionate were selected, with shaking, to mimic an intracellular environment for a typical MTb bacterium sequestered inside a cell in the body. Accordingly, in addition to showing that certain VOCs may consistently identify particular bacteria despite varying media and/or source conditions, this data can be used in a VOC data library for samples obtained directly from the body, for example from the breath of an individual.

### 4.4 MTb VOC Expression in Different Concentrations of Propionate

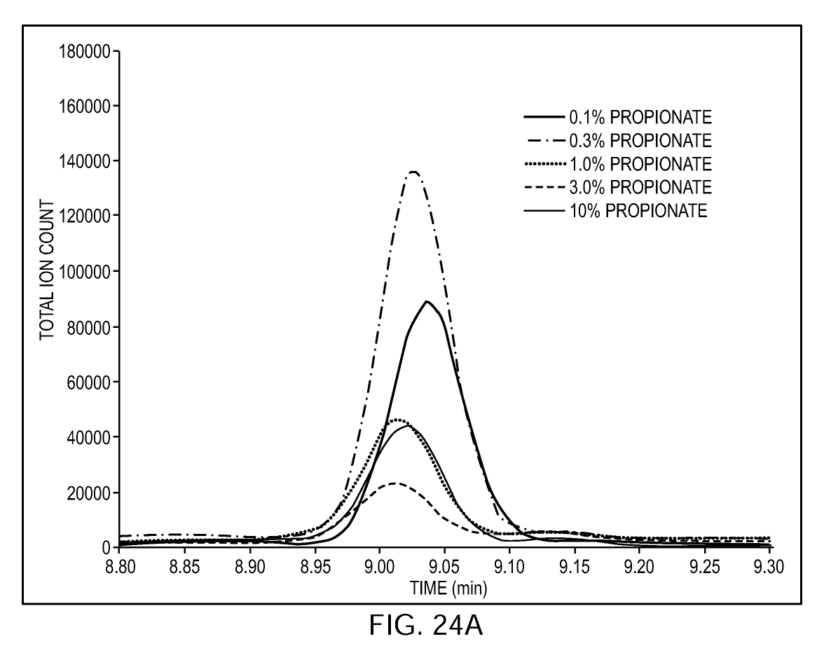

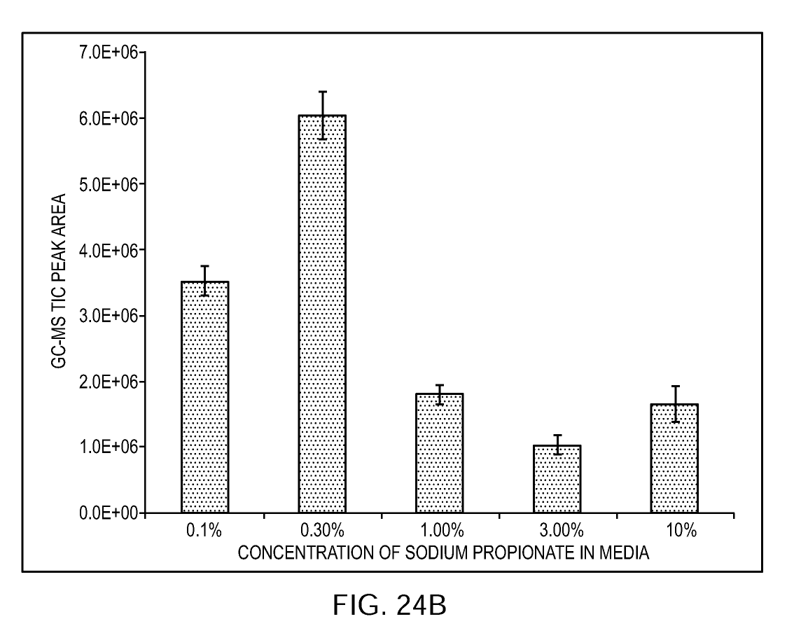

In another set of experiments, MTb was cultured with media containing sodium propionate at concentrations ranging from 0.1% to 10%. The extracted VOCs were detected using methods described above. The quantities of certain VOCs (methyl propionate, 3-pentanone, methyl 2-methylpropionate, and methyl 2-ethyl hexanoate) extracted were dependent on the propionate concentration in the media. The effect of media concentration on the expression of methyl propionate is depicted in FIGS. 24A and 24B.

### 4.5 MTb VOC Expression Cultured in Different Combinations of Lipids

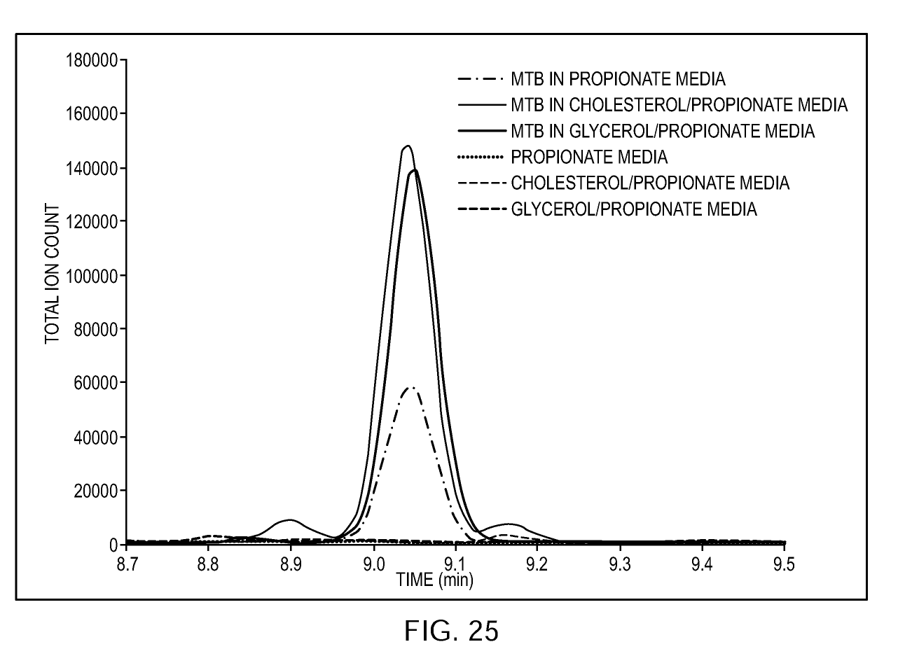

In another experiment, MTb was cultured on combinations of media containing one or more carbon sources, namely: propionate (0.3%), glycerol (0.1%), and/or cholesterol (0.01%). The extracted VOCs were detected using methods described above. Quantities of certain VOCs (methyl propionate, 3-pentanone, methyl 2-methylpropionate, and methyl 2-ethyl hexanoate) extracted were dependent on the contents of the media. In one case, the amount of methyl propionate expressed by the MTb was greatly increased in media containing both propionate and either glycerol or cholesterol, as depicted in the GC-MS total ion chromatogram in FIG. 25.

### Example 5

These experiments show that one or more VOCs may be used to identify the state (e.g. viable, growing, etc.) of a particular bacteria in the sample. In particular, MTb cultures were exposed to antibiotic treatment and the VOCs were detected. The resulting VOC data is shown in Table Z.

TABLE Z
MTb VOCs after exposure to antibiotics

| | | RV + | RV + | | | Confidence |
|---|---|---|---|---|---|---|
| Compound | CAS # | INH | RIF | RV | MEDIA | in ID |
| 1-ethoxy-2- | 627-02-1 | + | + | − | − | low |
| methylpropane | | | | | | |
| 1-ethoxybutane | 628-81-9 | + | + | − | − | low |
| t-butyl ethyl ether | 637-92-3 | + | + | − | − | medium |
| isobutanol | 78-83-1 | + | − | − | − | medium |
| methyl propionate | 554-12-1 | + | + | + | − | high |
| 3-pentanone | 96-22-0 | + | + | + | − | high |
| methyl 2-methyl | 868-57-5 | − | + | − | − | medium |
| butanoate | | | | | | |
| methyl-2-ethyl | 816-19-3 | + | + | + | − | high |
| hexanoate | | | | | | |

The results in Table AA show that effective antibiotic treatment of MTb may be identified via VOC detection.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing form the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method for identifying Staphylococcus aureus bacteria in a sample, the method comprising:
a. collecting a sample suspected of comprising Staphylococcus aureus bacteria; and
b. detecting one or more volatile organic compounds indicative of a presence of or response to treatment or resistance of the Staphylococcus aureus bacteria in the sample, the one or more volatile organic compounds being selected from the group consisting of methanethiol, dimethyl sulfide, 2,3-butanedione, 3-hydroxy-2-butanone, butyl acetate, and benzeneacetaldehyde.

2. The method of claim 1, wherein the one or more volatile organic compounds comprises methanethiol.

3. The method of claim 1, wherein the one or more volatile organic compounds comprises dimethyl sulfide.

4. The method of claim 1, wherein the one or more volatile organic compounds comprises 2,3-butanedione.

5. The method of claim 1, wherein the one or more volatile organic compounds comprises 3-hydroxy-2-butanone.

6. The method of claim 1, wherein the one or more volatile organic compounds comprises butyl acetate.

7. The method of claim 1, wherein the one or more volatile organic compounds comprises benzeneacetaldehyde.

8. The method of claim 1, wherein a combination of two or more volatile organic compounds is indicative of the presence of or response to treatment or resistance of Staphylococcus aureus in the sample.

9. The method of claim 1, wherein the sample was exposed to a candidate therapy for treating Staphylococcus aureus.

10. The method of claim 1, wherein the sample comprises exhaled breath from an individual.

11. The method of claim 1, wherein the sample is selected from the group consisting of sputum, blood, urine, pleural fluid, and pleural biopsy tissue.

12. The method of claim 1, wherein an amount of one or more volatile organic compounds is detected.

13. The method of claim 1, wherein the detecting is performed using a differential mobility spectrometer.

14. A method for identifying Klebsiella pneumonia bacteria in a sample, the method comprising:
a. collecting a sample suspected of comprising Klebsiella pneumonia bacteria; and
b. detecting one or more volatile organic compounds indicative of a presence of or response to treatment or resistance of the Klebsiella pneumonia bacteria in the sample, the one or more volatile organic compounds being selected from the group consisting of methanethiol, 2-heptanone, 2-nonanone, and 2-undecanone.

15. The method of claim 14, wherein the one or more volatile organic compounds comprises methanethiol.

16. The method of claim 14, wherein the one or more volatile organic compounds comprises 2-heptanone.

17. The method of claim 14, wherein the one or more volatile organic compounds comprises 2-nonanone.

18. The method of claim 14, wherein the one or more volatile organic compounds comprises 2-undecanone.

19. The method of claim 14, wherein a combination of two or more volatile organic compounds is indicative of the presence of or response to treatment or resistance of Klebsiella pneumonia in the sample.

20. The method of claim 14, wherein the sample was exposed to a candidate therapy for treating Klebsiella pneumonia.

21. The method of claim 14, wherein the sample comprises exhaled breath from an individual.

22. The method of claim 14, wherein the sample is selected from the group consisting of sputum, blood, urine, pleural fluid, and pleural biopsy tissue.

23. The method of claim 14, wherein an amount of one or more volatile organic compounds is detected.

24. The method of claim 14, wherein the detecting is performed using a differential mobility spectrometer.

25. A method for identifying Escherichia coli bacteria in a sample, the method comprising:
a. collecting a sample suspected of comprising Escherichia coli bacteria; and
b. detecting one or more volatile organic compounds indicative of a presence of or response to treatment or resistance of Escherichia coli bacteria in the sample, the one or more volatile organic compounds being selected from the group consisting of methanethiol and dimethyl disulfide.

26. The method of claim 25, wherein the one or more volatile organic compounds comprises methanethiol.

27. The method of claim 25, wherein the one or more volatile organic compounds comprises dimethyl disulfide.

28. The method of claim 25, wherein a combination of two or more volatile organic compounds is indicative of the presence of or response to treatment or resistance of Escherichia coli in the sample.

29. The method of claim 25, wherein the sample was exposed to a candidate therapy for treating Escherichia coli.

30. The method of claim 25, wherein the sample comprises exhaled breath from an individual.

31. The method of claim 25, wherein the sample is selected from the group consisting of sputum, blood, urine, pleural fluid, and pleural biopsy tissue.

32. The method of claim 25, wherein an amount of one or more volatile organic compounds is detected.

33. The method of claim 25, wherein the detecting is performed using a differential mobility spectrometer.
